(19) United States
(12) Patent Application Publication
(10) Pub. No.: US 2004/0122089 A1
(43) Pub. Date: Jun. 24, 2004

(54) Novel eicosanoid analgesics

(76) Inventors: Billy R. Martin, Richmond, VA (US); Raj K. Razdan, Gloucester, MA (US); Vincenzo Di Marzo, Portici (IT)

Correspondence Address: WHITHAM, CURTIS & CHRISTOFFERSON, P.C., 11491 SUNSET HILLS ROAD, SUITE 340, RESTON, VA, 20190, US

(21) Appl. No.: 10/365,607

(22) Filed: Feb. 13, 2003

Related U.S. Application Data
(63) Continuation-in-part of application No. 10/170,204, filed on Jun. 13, 2002.
(60) Provisional application No. 60/299,199, filed on Jun. 20, 2001.

(51) Int. Cl. C11C 3/00; A61K 31/277; A61K 31/16

(52) U.S. Cl. 514/509; 514/521; 514/627; 554/51; 554/54

(57) ABSTRACT

Analogs of andandamide and arvanil have been found to act preferential at CB₁ and AR₁ receptors, and at receptors other than CB₁ and AR₁. The analogs provide analgesic effects in vivo, and are useful in pain management. In addition, the analogs may be used as anti-proliferative/anti-tumor agents, vasodilators, and in other applications. Several of the anandamide and arvanil analogs are more potent than anandamide and arvanil.

### CROSS-REFERENCE TO RELATED APPLICATIONS

This application is a continuation in part of pending United States patent application Ser. No. 10/170,204, filed Jun. 13, 2002, the complete contents of which is hereby incorporated by reference.

This invention arose while performing research under grants from the National Institutes of Health (NIH) DA 09789, DA 07027, and DA 08904. The U.S. Government may have certain rights in the invention.

### DESCRIPTION

### BACKGROUND OF THE INVENTION

1. Field of the Invention

The invention is directed to certain analogs of anandamide and arvanil which may be useful as analgesics, anti-inflammatory compounds, vasodilators, and antiproliferative pharmaceutials, and as probes in biochemical studies on CB₁ or VR₁ receptors. More particularly, the invention pertains to compounds which are analogs of anandamide or arvanil which are either (i) highly selective to the CB₁ receptor relative to the VR₁ receptor, or vice-versa, (ii) active at both the CB₁ and VR₁ receptors, or (iii) are active at a receptor different from the CB₁ and VR₁ receptors.

2. Background Description

Eicosanoids are defined as any of a number of biochemically active compounds resulting from enzymic oxidation of arachidonic acid, e.g., prostaglandins, thromboxanes, prostacycline, and leukotrienes. As a group, they comprise what is often referred to as the arachidonic acid cascade.

Anandamide (arachidonoylethanolamide, AEA) is a putative endogenous agonist at cannabinoid CB₁ receptors. AEA binds with moderate affinity to CB₁ receptors (Ki 22-143 nM) and exhibits a pharmacological profile similar but not identical to that of (−)−Δ⁹-tetrahydrocannabinol (THC), the best studied plant cannabinoid. AEA is also a full agonist at the capsaicin receptor, which is a ligand and heat-activated non-selective cation channel named “vanilloid” receptor type 1 (VR₁). The potency of AEA in functional assays of VR₁-mediated activity (EC₅₀ 1-5 μM) in Xenopus oocytes or human embryonic kidney (HEK) cells over-expressing either rat or human VR₁ is ten to twenty fold lower than that reported for AEA activation of CB₁ receptors. Apart from CB₁, CB₂, and VR₁ receptors, AEA was shown to directly interact with binding sites distinct from either CB₁ or CB₂ receptors in endothelial cells, mouse and rat astrocytes and mouse brain. The selective antagonist of CB₁ receptors SR14176A does not block the typical cannabimimetic effects of AEA in the mouse ‘tetrad’ of tests, which include hypothermia, suppression of spontaneous activity, immobility on a ring, and analgesia in the tail-flick test. Furthermore, these effects of AEA are still observed in mutant mice where the CB₁ gene has been disrupted (“CB 1 knockouts”). Therefore, it is likely that the molecular targets of AEA are not confined to CB₁ receptors. Since VR₁ is expressed in several brain regions, it is possible that this receptor is partly responsible for some of the neurobehavioral effects of AEA. Moreover, AEA activates G-protein-coupled receptors (CPGRs) in brain membranes prepared from CB1 knockouts, thus suggesting the existence of non-CB₁, non-CB₂, non-VR₁ receptors through with AEA may produce some of its pharmacological effects.

Arvanil (N-[3-methoxy-4-hydroxy-benzyl]-arachidonamide) is a structural “hybrid” between the endogenous cannabinoid CB₁ receptor ligand AEA and capsaicin. Arvanil has an affinity for CB₁ receptors comparable to AEA, and activates GPCRs, including CB₁ receptors, in mouse brain membranes as potently as, but less efficaciously than, AEA. It also activates VR₁ receptors more potently than AEA and capsaicin, is more resistant to enzymatic hydrolysis than AEA, and is a potent inhibitor of AEA facilitated transport into cells. Arvanil is much more potent than either AEA or capsaicin (I) as an antiproliferative agent for human breast cancer cells, in a fashion sensitive to both CB₁ and VR₁ receptor antagonists, (ii) as a cannabimimetic agent in the mouse ‘tetrad’, in a fashion insensitive to SR141716A, (iii) as a spinal analgesic insensitive to either SR141716A or the VR₁ antagonist capsazepine, and (iv) as a relaxant of mouse vas deferens.

However, studies have shown that aravanil efficacy is not well balanced between VR₁ and CB₁ receptors. Hence, it would be advantageous to have alternative “hybrid” CB₁/VR₁ agonists which either have highly selective activity at either of CB₁ or VR₁ receptors, activity at both CB₁ and VR₁receptors, or activity at a different receptor in the brain, or are active at other receptor sites in the brain, spine, etc.

(R)-1′-Methyl-2′-hydroxy-ethyl-arachidonamide (Met-AEA) is a metabolically stable AEA analog exhibiting higher affinity for CB₁ receptors, and higher potency and efficacy in vivo than AEA. Also, Met-AEA activates vanilloid receptors ex vivo and is approximately 100 fold more selective for CB₁ versus VR₁ receptors. AEA analogs obtained by branching and elongating the alkyl chain in AEA and Met-AEA, such as dimethyl-heptyl (DMH) derivatives are also more potent as CB₁ receptor ligands than AEA (however, the selectivity to VR₁ was not tested prior to this invention).

### SUMMARY OF THE INVENTION

It is an object of this invention to provide novel eicosanoid analgesic compounds useful in analgesic, anti-tumor, vasodilator, and other applications, and for use in research applications to assess the involvement of CB₁, VR₁, and other receptors.

According to the invention, anandamide and arvanil analogs have been identified which are effective agonists of CB₁, VR₁ and other binding sites (i.e., non-CB₁, non-CB₂, non-VR₁ binding sites). The compounds can be used as selective probes for biochemical studies on CB₁ or VR₁ receptors, or as novel analgesic, anti-inflammatory, vasodilator, and anti-proliferative drugs, or as templates for the same. The compounds should have particular application in the treatment of acute and chronic pain, migraine and in inflammation.

### BRIEF DESCRIPTION OF THE DRAWINGS

FIG. 1. Synthesis scheme: (a) CuI, NaI, K₂CO₃, 2, DMF, 18 h, 23° C., 87%; (b) CBr₄, PPh₃, CH₂Cl₂, −20° C. to 23° C., 1 h, 92%; (c) CuI, NaI, K₂CO₃, 4, DMF, 18h, 23° C., 85%; (d) P-2Ni, Ethanol, 3 h, 23° C., 50%.

FIG. 2. Synthesis scheme: (a) PPh₃, Imidazole, Ether/CH₃CN, 0° C. to 23° C., 1 h, 98%; (b) PPh₃, CH₃CN, reflux, 18 h, 90%; (c) NHMDS, THF/HMPA, 9, −78° C. to 23° C., 2 h, 61%; (d) LiOH, MeOH/H₂O, 23° C., 18 h, 94%; (e) Oxalyl chloride, CH₂Cl₂, 0° C., 2 h, 100%; (f) Vanillylamine, CH₂Cl₂, 0° C. to 23° C., 18 h, 51%.

FIG. 3. Synthesis scheme: NHMDS, THF/HMPA, 11, −78° C. to 23° C., 2 h, 50%; (b) LiOH, MeOH/H₂O, 23° C., 18 h, 94%; (c) EDCI, DMAP, Vanillylamine, CH₂Cl₂, 0° C. to 23° C., 18 h, 26%.

FIG. 4. Synthesis scheme: (a) LDA, THF/HMPA, −78° C. to 23° C., 2 h, 88%; (b) 9-BBN, THF, Ethanol/6N NaOH/H2O2, 50° C., 1 h, 83%; (c) DHP, PPTS, CH₂Cl₂, 23° C., 4 h, 92%; (d) LAH, Ether, 0° C., 0.5 h, 95%; (e) PCC, CH₂Cl₂, 23° C., 2 h, 79%.

FIG. 5. Synthesis scheme: (a) Br₂, PPh₃, CH₂Cl₂, 0° C. to 23° C., 18 h, 50%; (b) KCN, DMSO, 50° C., 5 h, 78%; c) LiOH, MeOH/H₂O, 23° C., 18 h, 94%; (d) Oxalyl chloride, CH₂Cl₂, 0° C., 2 h, 100%; (e)Vanillylamine, CH₂Cl₂, 0° C. to 23° C., 18 h, 32-28%.

### DETAILED DESCRIPTION OF THE PREFERRED EMBODIMENTS OF THE INVENTION

Anandamide and arvanil analogs have been prepared and tested as set forth in Examples 1-4 below, and have been found to be effective agonists of CB₁, VR₁ and other binding sites (i.e., non-CB₁, non-CB₂, non-VR₁ binding sites). All except for seven of the compounds fall within the following three general categories:

Category 1:

Compounds within this category have the following general structure:

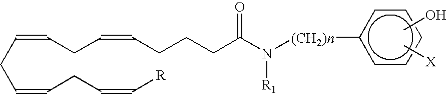

where n ranges from 0-5; X represents a hydrogen, C₁₋₆ alkyl, a C₁₋₆ O-alkyl (e.g., methoxy (OCH₃), ethoxy (OEt), etc.) halogen (Cl, Br, I, F), and hydroxy (OH); R₁ represents hydrogen or C₁₋₆ alkyl; and R is represented by the chemical structure

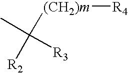

where m ranges from 1-7; R₂ and R₃ represent a hydrogen or C₁₋₆ alkyl group and may be the same or different from each other; and R₄ represents hydrogen, hydroxy, halogen, cyano (CN), C₁₋₆ alkyl (e.g., methyl (CH₃)), ONO, ONO₂, and NO₂.

Compounds synthesized and tested within this category include:

| Compound | Structure |
|---|---|
| O-1839 | 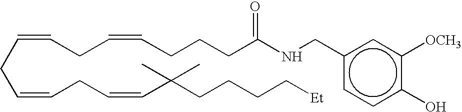 |
| O-1856 | 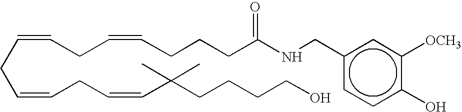 |
| O-1895 | 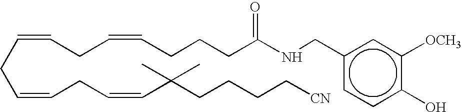 |
| O-1861 | 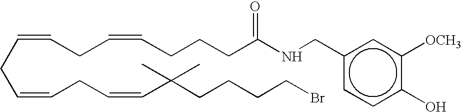 |
| O-1986 | 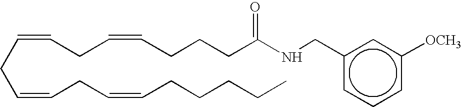 |
| O-2094 | 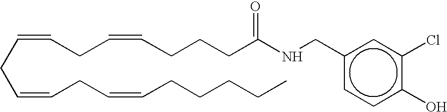 |
| O-1988 | 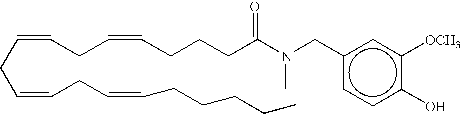 |

Category 2:

Compounds within this category have the general structure:

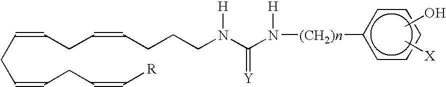

where n ranges from 0-5; X represents a hydrogen, C₁₋₆ alkyl, a C₁₋₆ O-alkyl (e.g., methoxy, ethoxy, etc.) halogen (Cl, Br, I, F), hydroxy (OH); Y represents S or O; and R is represented by the chemical structure

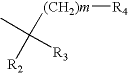

where m ranges from 1-7; R₂ and R₃ represent a hydrogen or C₁₋₆ alkyl group and may be the same or different from each other; and R₄ represents hydrogen, hydroxy, halogen, cyano (CN), C₁₋₆ alkyl (e.g., methyl (CH₃)), ONO, ONO₂, and NO₂.

Compounds synthesized and tested within this category include:

| Compound | Structure |
|---|---|
| O-1987 | 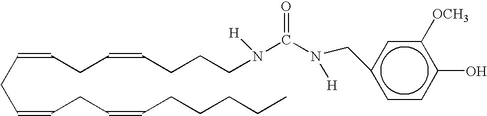 |
| O-2095 | 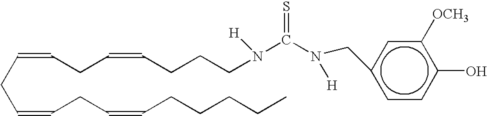 |
| O-2109 | 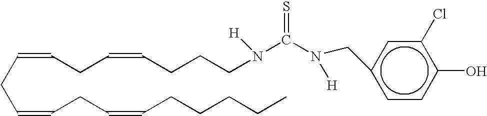 |

Other compounds which have been synthesized and tested include the following AEA analogs which have been methylated at the C16 position as well as forming a hydroxy, cyano, and halogen derivative:

| | |
|---|---|
| O-1811 | 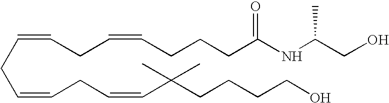 |
| O-1812 | 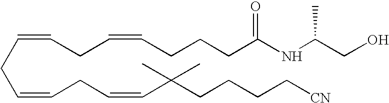 |
| O-1860 | 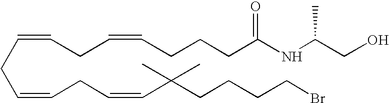 |

Methylated AEA derivatives with a derivatized tail of halogen, hydroxy, lower alkoxy, and cyano may also be considered a “category” of compounds useful in the practice of the invention.

The following two compounds, which are similar to but do not fall within any of the categories specified above may also be useful within the practice of this invention as analgesics, anti-inflammatories, vasodilators, and anti-proliferative drugs, or as templates for the same. The compounds should be useful in the treatment of acute and chronic pain, migraine and in inflammation.

| Compound | Structure |
|---|---|
| O-2142 | 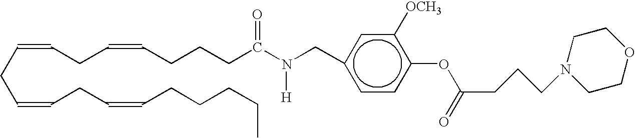 |
| O-2140 | 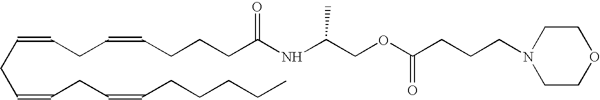 |

Based on the in vitro and in vivo results noted in Examples 2-4, the compounds of the present invention should be useful in a variety of analgesia applications including in pain management for acute and chronic pain (e.g., arthritis, migraine headache, tooth ache, inflammation from injuries or from surgery, etc.). The invention may also be used for vasodilation, and in anti-proliferative/anti-tumor or anti-cancer applications. The amount of the compound to be delivered will depend on the patient and the matter being treated. These compounds may be provided by intravenous injection, as is done in Examples 2 and 3, but other routes of delivery should also be suitable, including without limitation intradermal injection, subcutaneous injection, intramuscular injection, intraperitoneal injection; oral, rectal and buccal delivery; transdermal delivery; inhalation; etc. The compounds may be provided alone or in combination with other constituents, and may be provide in pure or salt form (e.g., hydrochloride salt, etc.). In addition, the compounds may be formulated with aqueous or oil based vehicles, and can be accompanied by preservative (e.g., methyl paraben or benzyl alkonium chloride (BAK)), surfactants (e.g.,oleic acid), solvents, elixirs, flavoring agents (in the case of oral delivery), and other materials (preferably those which are generally regarded as safe (GRAS)). The compounds may also be added to blood ex vivo and then be provided to the patient. Finally, as noted above, the compounds may also be used for research purposes, such as in probing for receptor sites and/or analyzing performance of various compounds at a patient/s receptor sits.

The following examples demonstrate that the anandamide and arvanil analogs of this invention are effective agonists of CB₁, VR₁ and other binding sites (i.e., non-CB₁, non-CB₂, non-VR₁ binding sites).

### EXAMPLE 1

Synthesis of N-Vanillyl-arachidonoyl-amide (Arvanil) and its Analogs, and procedure for synthesis of the synthon Methyl 14-Hydroxy-(all-cis)-5,8,11-Tetradecatrienoate.

All reagents were of commercial quality, reagent grade, and used without further purification. Anhydrous solvents were purchased from Aldrich and used without further purification. All reactions were carried out under N₂ atmosphere. ¹H NMR spectra were recorded on a JEOL Eclipse 300 spectrophotometer using CDCl₃ as the solvent with tetramethylsilane as an internal standard. Thin-layer chromatography (TLC) was carried out on Baker Si 250F plates and was developed upon treatment with phosphomolybdic acid (PMA). Flash column chromatography was carried out on EM Science silica gel 60. Elemental analyses were performed by Atlantic Microlab, Inc., Atlanta, Ga., and were found to be within ±0.4% of calculated values for the elements shown, unless otherwise noted.

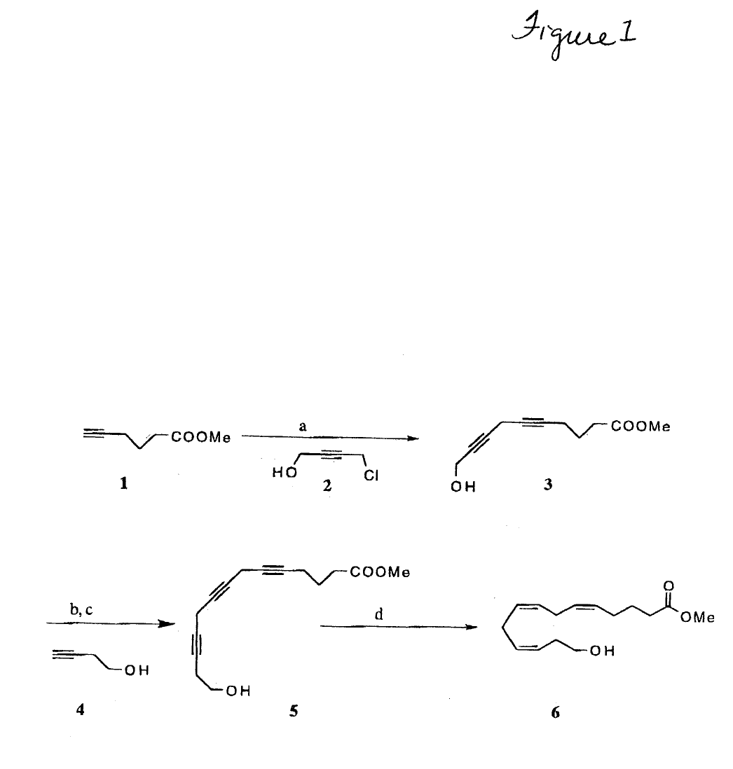

Methyl hex-5-ynoate 1 (FIG. 1): A stirred solution of hex-5-ynoic acid (5 g, 44.6 mmol), p-TSA (58 mg, 0.3 mmol) in MeOH (8 mL) and CH₂Cl₂ (17 mL) was refluxed for 24 hours. The mixture was quenched with saturated NaHCO₃ and the organic layer was separated. The aqueous layer was extracted with CH₂Cl₂. The combined organic layers were dried over MgSO₄ and evaporated under reduced pressure to yield the methyl ester (5.46 g, 97%). ¹H NMR δ1.84 (quint, 2H, J=7.2 Hz), 1.96 (t, 1H, J=2.75 Hz), 2.25 (dt, 2H, J=7.2, 2.75 Hz), 2.45 (t, 2H, J=7.2 Hz), 3.67 (s, 3H).

4-Chloro-but-2-yn-1-ol 2 (FIG. 1): To a stirred solution of but-2-yn-1,4-diol (86 g, 1 mol) and pyridine (89 mL, 1.1 mol) in benzene (100 mL) was added dropwise thionyl chloride (80.23 mL, 1.1 mol) over a period of 6 hours, while the temperature was maintained between 10°-20° C. The reaction mixture was then stirred overnight at room temperature. The mixture was poured into ice water (250 mL) and the benzene layer was separated. The aqueous layer was extracted with ether (4×100 mL) and the combined organic layers were washed with saturated NaHCO₃, water. The ether extract was dried over MgSO₄ and then removed under vacuum. Purification of the residual oil by distillation (80° C., 5 mmHg) provided the title compound as a colorless liquid (34.5 g, 33%). ¹H NMR δ4.18 (t, 2H, J=1.9 Hz), 4.33 (dt, 2H, J=6.3, 1.9 Hz).

10-Hydroxy-deca-5,8-diynoic acid methyl ester 3 (FIG. 1): A mixture of K₂CO₃ (5.94 g, 43 mmol), CuI (4.1 g, 22 mmol), 4-chloro-but-2-yn-1-ol 2 (FIG. 1) (4.47 g, 43 mmol), NaI (6.44 g, 43 mmol) and methyl hex-5-ynoate (5.46 g, 43 mmol) in DMF (86 mL) was stirred overnight at room temperature. The mixture was diluted with ethyl acetate and plugged through a pad of celite. It was washed with saturated NH₄Cl and brine. The solution was dried over MgSO4 and evaporated under vacuum. The oily residue was dissolved in hexanes/ethyl acetate (1/1) and plugged through a pad of silica gel to provide a yellowish oil (7.2 g, 87%) which was used without further purification. Some of the diyne decomposed upon flash chromatography. ¹H NMR δ1.81 (quint, 2H, J=6.7 Hz), 2.23 (tt, 2H, J=6.7, 2.2 Hz), 2.43 (t, 2H, J=7.4 Hz), 3.17 (quint, 2H, J=2.2 Hz), 3.67 (s, 3H), 4.25 (dt, 2H, J=6, 2.2 Hz).

14-Hydroxy-tetradeca-5,8,11-triynoic acid methyl ester 5 (FIG. 1): To a stirred solution of diyne 3 (8.65 g, 44.6 mmol) and CBr₄ (17.74 g, 53.5 mmol) in CH₂Cl₂ (80 mL) cooled −20° C. was added dropwise a solution of triphenylphosphine (14.6 g, 55.7 mmol) in CH₂Cl₂ (40 mL). After addition the cooling bath was removed and the mixture was stirred for an additional 1 hour. Hexanes/ethyl acetate (4/1) was then added until triphenylphosphine oxide precipitated. The mixture was plugged through a pad of silica gel to yield methyl 1 0-bromo-deca-5,8-diynoate as a colorless oil (10.54 g, 92%). Attempts to further purify by flash chromatography resulted in partial decomposition of the bromide. Hence, it was used as such in the subsequent reaction. ¹H NMR δ1.81 (quint, 2H, J=7.1 Hz), 2.23 (tt, 2H, J=7.1, 2.5 Hz), 2.43 (t, 2H, J=7.1 Hz), 3.20 (quint, 2H, J=2.5 Hz), 3.67 (s, 3H), 3.90 (t, 2H, J=2.5 Hz).

A mixture of K₂CO₃ (9.67 g, 70 mmol), CuI (6.66 g, 35 mmol), but-3-yn-1-ol 4 (5.3 mL, 70 mmol) (FIG. 1), NaI (10.50 g, 70 mmol) and methyl 10-bromo-deca-5,8-diynoate (17.99 g, 70 mmol) in DMF (140 mL) was stirred overnight at room temperature. The mixture was diluted with ethyl acetate and plugged through a pad of celite. It was washed with saturated NH₄Cl and brine. The solution was dried over MgSO₄ and evaporated under vacuum. The oily residue was dissolved in hexanes/ethyl acetate (1/1) and plugged through a pad of silica gel to provide compound 5 (FIG. 1) as a yellowish oil (14.63 g, 85%) which was used without further purification. Attempts to purify by flash chromatography resulted in partial decomposition of the triyne. ¹H NMR δ1.80 (quint, 2H, J=6.7 Hz), 2.23 (tt, 2H, J=6.9, 2.2 Hz), 2.42 (t, 2H, J=7.1 Hz), 2.42-2.48 (m, 2H), 3.14 (dq, 4H, J=6.3, 2 Hz), 3.67 (s, 3H), 3.69 (t, 2H, J=6 Hz).

14-Hydroxy-tetradeca-all-cis-5,8,11-trienoic acid methyl ester 6 (FIG. 1): To a stirred solution of Ni(OAc)₂ (14.93 g, 60 mmol) in EtOH (450 mL) was added ethylenediamine (4 mL, 60 mmol) followed by a 1 M solution of NaBH₄ (60 mL). The mixture was stirred at room temperature for 30 minutes. The triyne 5 (6.6 g, 26.8 mmol) was added to the reaction mixture and a H₂ atmosphere (balloon) was kept over the reaction mixture. It was stirred for 3 hours at room temperature and the solvent was removed in vacuo. The residue was dissolved in hexanes/ethyl acetate (1/1) and plugged through a pad of silica gel. Purification by flash chromatography (eluting with hexanes/ethyl acetate 2/1) provided the desired triene 6 (FIG. 1) as a colorless oil (3.38 g, 50%). ¹H NMR δ1.70 (quint, 2H, J=7.1 Hz), 1.98-2.14 (m, 2H), 2.27-2.39 (m, 2H), 2.32 (t, 2H, J=7.1 Hz), 2.79 (dd, 2H, J=5.2, 5.2 Hz), 2.84 (dd, 2H, J=5.8 Hz), 3.65 (t, 2H, J=6.6 Hz), 3.66 (s, 3H), 5.29-5.58 (m, 6H).

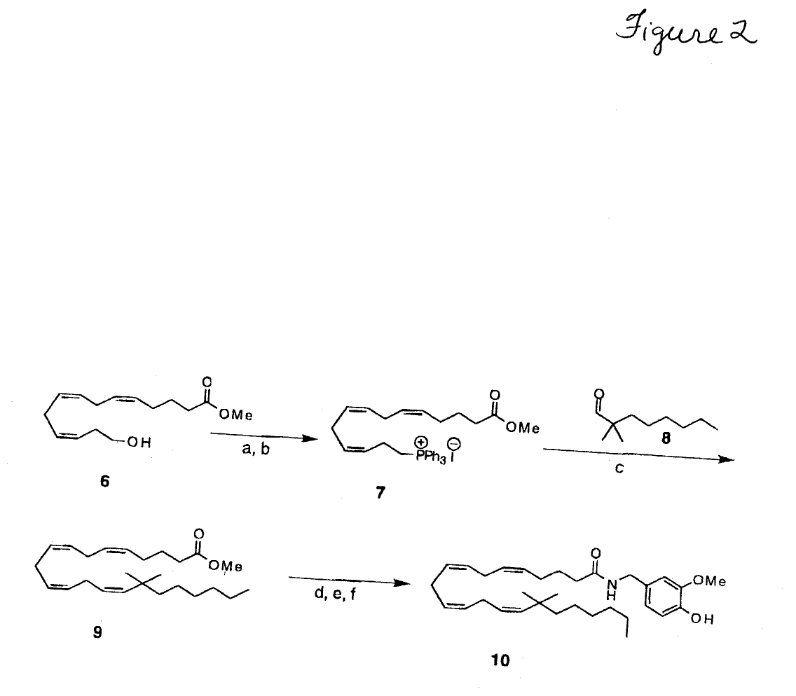

Methyl 14-triphenylphosphonio-tetradeca-all-cis-5,8,11-trienoate iodide 7 (FIG. 2): To a stirred solution of triphenylphosphine (456 mg, 1.74 mmol) and imidazole (118 mg, 1.74 mmol) in Et₂O/CH₃CN (5 mL/1.7 mL) cooled to 0° C. was added I₂ (441 mg, 1.74 mmol) in several portions. The resulting slurry was warmed to room temperature and stirred for 20 minutes. It was cooled to 0° C. and the alcohol 6 (FIG. 2) was added slowly. The mixture was warmed to room temperature after addition and stirred for 1 hour ar room temperature. It was diluted with pentane/ether (4/1) and plugged through a pad of silica gel to yield the iodide as a colorless oil (562 mg, 98%). ¹H NMR δ1.70 (quint, 2H, J=7.4 Hz), 1.95-2.17 (m, 4H), 2.32 (t, 2H, J=7.4 Hz), 2.66 (q, 2H, J=7.4 Hz), 2.80 (m, 2H), 3.15 (t, 2H, J=7.2 Hz), 3.66 (s, 3H), 5.29-5.58 (m, 6H).

A solution of triphenylphosphine (2.25 g, 8.6 mmol) and the iodide (2.83 g, 7.82 mmol) in acetonitrile (50 mL) was refluxed overnight. The solvent was removed under reduced pressure and the oily residue was purified by washing and decanting with hexanes/benzene (1/1; 80 mL). The solvent was removed and the oily residue was heated in a vacuum oven overnight at 60° C. to yield 7 (FIG. 2) as a yellow gum (90%) which was used without further purification. ¹H NMR δ1.66 (quint, 2H, J=7.4 Hz), 2.04 (q, 2H, J=7.4 Hz), 2.29 (t, 2H, J=7.4 Hz), 2.39-2.52 (m, 2H), 2.58 (t, 2H, J=7.2 Hz), 2.63 (t, 2H, J=6.6 Hz), 3.64 (s, 3H), 3.80-3.90 (m, 2H), 5.14-5.67 (m, 6H), 7.64-7.73 (m, 6H), 7.78-7.88 (m, 9H).

16,16-Dimethyl-docosa-5,8,11,14-all-cis-tetraenoic acid methyl ester 9 (FIG. 2): To a stirred solution of the phosphonium salt 7 (686 mg, 1.1 mmol) in THF/HMPA (6 mL/1 mL) cooled to −10° C. was added dropwise a 1M solution of NHMDS in THF (1.1 mL). The mixture was stirred at 0° C. for 30 minutes and cooled to −78° C. The aldehyde 8 (171 mg, 1.1 mmol) (FIG. 2) was added dropwise in THF (1.5 mL) to the reaction mixture. The cooling bath was removed and it was left to warm to room temperature over 2 hours. It was quenched with hexanes and the mixture was plugged through a pad of silica gel (eluting with ethyl acetate/hexanes 1/4). The filtrate was dried over MgSO₄ and the solvent was removed under reduced pressure. The oily residue was purified by flash chromatography (3% EtOAc/97% hexanes) to yield the tetraene 9 (FIG. 2) as a colorless oil (0.25 g, 61%). ¹H NMR δ0.87 (t, 3H, J=6.6 Hz), 1.09 (s, 6H), 1.18-1.35 (m, 10H), 1.70 (quint, 2H, J=7.4Hz), 2.00-2.11 (m, 2H), 2.32 (t, 2H, J=7.4 Hz), 2.70-2.91 (m, 6H), 3.66 (s, 3H), 5.12-5.25 (m, 2H), 5.30-5.41 (m , 6H).

16,16-Dimethyl-docosa-5,8,11,14-all-cis-tetraenoic acid (4-hydroxy-3-methoxy-benzyl) amide 10 (O-1839; FIG. 2): To a stirred solution of ester 9 (250 mg, 0.67 mmol) (FIG. 2) in MeOH (33 mL) and water (11 mL) was added LiOH.H₂O (190 mg, 4.8 mmol) and the reaction mixture was stirred at 55° C. overnight. It was diluted with ether and acidified with 10% HCl. The layers were separated and the aqueous layer was extracted with ether. The combined organic layers were dried over MgSO₄ and evaporated to yield the crude acid (224 mg, 93%) which was used directly. The acid (224 mg, 0.62 mmol) was dissolved in CH₂Cl₂ (7 mL) and cooled to 0° C. A 2M solution of oxalyl chloride in CH₂Cl₂ (0.67 mL) was added dropwise followed by 2 drops of DMF. The ice bath was removed and the mixture was stirred at room temperature for 2 hours. The solvent was evaporated under vacuum. A solution of the acid chloride in CH₂Cl₂ (3 mL) was added to a solution of 4-hydroxy-3-methoxy benzylamine (1 g, 5.2 mmol) in CH₂Cl₂ (5 mL) at 0° C. The ice bath was removed and the mixture was stirred at room temperature overnight. It was diluted with CH₂Cl₂ and washed with brine. The organic layer was dried over MgSO₄ and then concentrated in vacuo. The crude product was purified by flash chromatography (hexanes/EtOAc 2/1) to yield the amide 10 (170 mg, 55%) (FIG. 2). ¹H NMR δ0.87 (t, 3H, J=6.9 Hz), 1.08 (s, 6H), 1.22-1.35 (m, 10H), 1.73 (quint, 2H, J=7.4 Hz), 2.00-2.12 (m, 2H), 2.20 (t, 2H, J=7.4 Hz), 2.77 (t, 2H, J=5.5 Hz), 2.81 (t, 2H, J=5.5 Hz), 2.92 (t, 2H, J=5.5 Hz), 3.87 (s, 3H), 4.34 (d, 2H, J=5.8 Hz), 5.12-5.25 (m, 2H), 5.30-5.41 (m , 6H), 5.60 (s, 1H), 5.63 (br s, 1H), 6.74-6.87 (m, 3H). Anal. Calcd. for C₃₁H₄₄O₃N₂.0.6H₂O: C, 75.87; H, 9.99. Found: C, 75.89; H, 9.89.

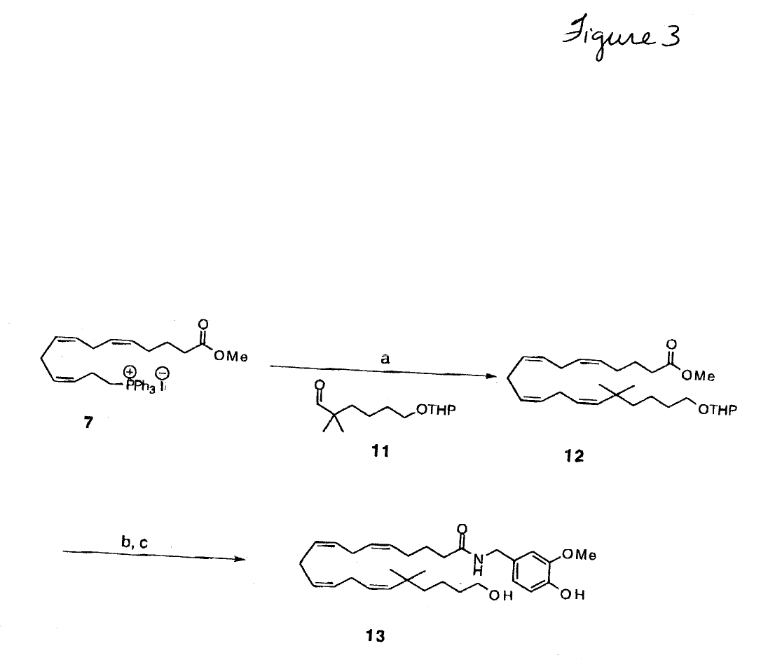

2,2-Dimethyl-(tetrahydro-pyran-6-yloxy)-hexanal 11 (FIG. 3):

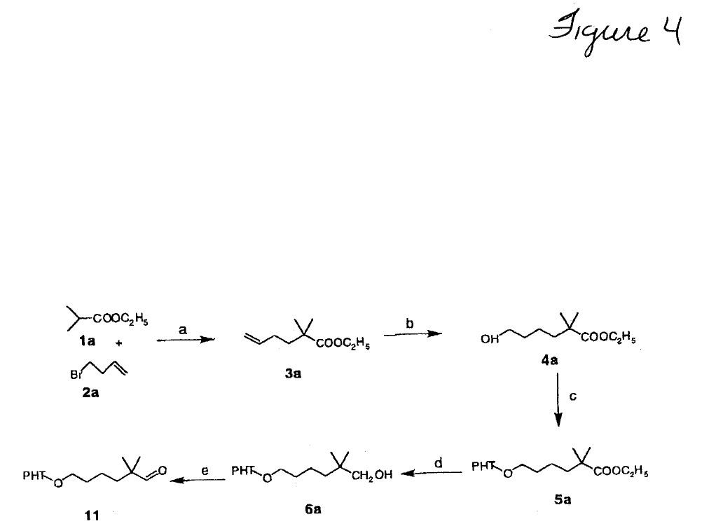

Ethyl 2,2-dimethyl-hex-5-enoate 3a (FIG. 4): To a stirred solution of diisopropylamine (5.7 mL, 41 mmol) in THF cooled to −78° C. was added dropwise a 2.5M solution of n-BuLi in hexanes (16.4 mL, 41 mL). The reaction mixture was stirred for 30 minutes and ethyl isobutyrate 1a (5.1 mL, 37 mmol) (FIG. 4) was added dropwise at −78° C. The mixture was stirred for 30 minutes and 4-bromo-but-1-ene 2a (5 g, 37 mmol) (FIG. 4) in HMPA (7.4 mL) was added dropwise. Upon addition, the mixture was left to warm to room temperature and was stirred for 2 hours. It was quenched with water and extracted with ethyl acetate. It was then washed with 10% HCl, saturated NaHCO₃ and dried over MgSO₄. The residual oil was plugged through a pad of silica gel (4/1 hexanes/EtOAc) to yield a slightly yellow oil (5.54 g, 88%). ¹H NMR δ1.17 (s, 6H), 1.24 (t, 3H, J=7.2 Hz), 1.56-1.63 (m, 2H), 1.94-2.02 (m, 2H), 4.10 (q, 2H, J=7.2 Hz), 4.92 (ddt, 1H, J=10.2, 1.9, 1.4 Hz), 4.99 (ddt, 1H, J=17, 1.9, 1.9 Hz), 5.78 (ddt, 1H, J=17, 10.2, 6.6 Hz).

Ethyl 2,2-dimethyl-6-hydroxy-hexanoate 4a (FIG. 4): To a stirred 0.5 M solution of 9-BBN in THF (65.2 mL, 32.6 mmol) was added ethyl 2,2-dimethyl-hex-5-enoate 3a (5.54 g, 32.6 mmol) (FIG. 4) in THF (16.2 mL). After 2 hours at room temperature, the reaction mixture was cooled to 0° C. Ethanol (20 mL) was added followed by 6N NaOH (6.5 mL) and 30% H₂O₂ (13 mL). The mixture was then heated to 50° C. and stirred for 1 hour. After cooling to room temperature, it was diluted with brine and extracted with EtOAc. The organic layer was dried and evaporated under vacuum. Purification of the oil by flash chromatography (1/1 hexanes/EtOAc) yielded the alcohol as a colorless oil (5.1 g, 83%). ¹H NMR δ1.17 (s, 6H), 1.21-1.34 (m, 4H), 1.24 (t, 3H, J=7.2 Hz), 1.49-1.55 (m, 2H), 3.63 (br s, 2H), 4.10 (q, 2H, J=7.2 Hz).

Ethyl 2,2-dimethyl-6-(tetrahydro-pyran-2-yloxy -hexanoate 5a (FIG. 4): A mixture of alcohol 4a (5.1 g, 27 mmol)(FIG. 4), PPTS (200 mg, 0.8 mmol) and DHP (2.96 mL, 32.4 mmol) in CH₂Cl₂ was stirred at room temperature for 4 hours. The mixture was diluted with water and extracted with CH₂Cl₂. The organic layer was dried over MgSO₄ and evaporated to yield the product as a colorless oil (6.75 g, 92%). ¹H NMR δ1.16 (s, 6H), 1.23-1.32 (m, 4H), 1.24 (t, 3H, J=7.2 Hz), 1.49-1.90 (m, 8H), 3.36 (dt, 1H, J=9.4, 6.6 Hz), 1.23-1.32 (m, 1H), 3.70 (dt, 1H, J=9.4, 6.6 Hz), 3.83-3.90 (m, 1H), 4.10 (q, 2H, J=7.2 Hz), 4.56 (br t, 1H, J=2.5 Hz).

2,2-Dimethyl-(tetrahydro-pyran-6-yloxy)-hexan-1-ol 6a (FIG. 4): To a suspension of LAH (943 mg, 24.8 mmol) in ether (120 mL) cooled to 0° C. was added the ester 5a (6.75 g, 24.8 mmol) in ether (10 mL) dropwise. After stirring for 30 minutes, the reaction was quenched by the careful addition of water. The aqueous layer was extracted with ether and the combined organic layers were dried and evaporated to provide the alcohol as a colorless oil (5.34 g, 95%). 1H NMR δ0.86 (s, 6H), 1.23-1.32 (m, 4H), 1.49-1.90 (m, 8H), 3.30 (dd, 2H, J=6.3, 1.9 Hz), 3.41 (dt, 1H, J=9.4, 6.6 Hz), 3.48-3.52 (m, 1H), 3.75 (dt, 1H, J=9.4, 6.6 Hz), 3.83-3.90 (m, 1H), 4.56 (br t, 1H, J=2.5 Hz).

2,2-Dimethyl-(tetrahydro-pyran-6-yloxy)-hexanal 11 (FIG. 4): To a stirred suspension of PCC (4.55 g, 21.45 mmol) and celite in CH₂Cl₂ (40 mL) was added a solution of the alcohol 6a (3.3 g, 14.3 mmol) (FIG. 4) in CH₂Cl₂ (10 mL). The mixture was stirred at room temperature for 2 hours. It was then filtered through a pad of silica gel (hexanes/EtOAc 3/1) and the solution was evaporated to yield the aldehyde as a colorless oil (3.6 g, 79%). ¹H NMR δ1.04 (s, 6H), 1.23-1.32 (m, 4H), 1.45-1.85 (m, 8H), 3.36 (dt, 1H, J=9.4, 6.6 Hz), 3.48-3.52 (m, 1H), 3.70 (dt, 1H, J=9.4, 6.6 Hz), 3.83-3.90 (m, 1H), 4.56 (br t, 1H, J=2.5 Hz), 9.44 (s, 1H).

20-(Tetrahydro-pyran-2-yloxy)-16,16-dimethyl-eicosa-5,8,11,14-all-cis-tetraenoic acid methyl ester 12 (FIG. 3): To a stirred solution of the phosphonium salt 7 (4.36 g, 6.98 mmol) (FIG. 3) in THF/HMPA (40 mL/5 mL) cooled to −10° C. was added dropwise a 1M solution of NHMDS in THF (6.98 mL). The mixture was stirred at 0° C. for 30 minutes and cooled to −78° C. The aldehyde 11 (1.58 g, 6.98 mmol) (FIG. 3) was added dropwise in THF (8 mL) to the reaction mixture. The cooling bath was removed and it was left to warm to room temperature over 2 hours. It was quenched with hexanes and the mixture was plugged through a pad of silica gel (eluting with ethyl acetate/hexanes 1/4). The filtrate was dried over MgSO₄ and the solvent was removed under reduced pressure. The oily residue was purified by flash chromatography (EtOAc/hexanes 1/4) to yield the tetraene 12 (FIG. 3) as a colorless oil (1.56 g, 50%). ¹H NMR δ1.10 (s, 6H), 1.25-1.40 (m, 4H), 1.45-1.63 (m, 8H), 1.70 (quint, 2H, J=7.4 Hz), 1.97-2.13 (m, 2H), 2.31 (t, 2H, J=7.4 Hz), 2.75-2.95 (m, 6H), 3.37 (dt, 1H, J=9.6, 6.6 Hz), 3.50 (br dt, 1H, J=11.3, 5.2 Hz), 3.66 (s, 3H), 3.72 (dt, 1H, J=9.6, 6.6 Hz), 3.86 (br dt, 1H, J=11.3, 5.2 Hz), 4.57 (br t, 1H, J=4.4 Hz), 5.12-5.41 (m, 8H).

16,16-Dimethyl-20-hydroxy-eicosa-5,8,11,14-all-cis-tetraenoic acid (4-hydroxy-3-methoxy-benzyl) amide 13 (O-1856; FIG. 3): To a stirred solution of tetraene 12 (300 mg, 0.67 mmol) (FIG. 3) in MeOH (33 mL) and water (11 mL) was added LiOH.H₂O (190 mg, 4.8 mmol) and the mixture was stirred at room temperature overnight. It was diluted with ether and acidified with 10% HCl. The layers were separated and the aqueous layer was extracted with ether. The combined organic layers were dried over MgSO₄ and evaporated to yield the crude acid (258 mg, 90%) which was used directly. The acid was added to a stirred solution of 4-hydroxy-3-methoxy benzylamine (358 mg, 1.89 mmol) pretreated with Et₃N, DMAP (93 mg, 0.76 mmol) and EDCI (146 mg, 0.76 mmol) in CH₂Cl₂ (5 mL) cooled to 0° C. The mixture was stirred for 30 minutes and the ice bath was removed. It was left to stir overnight at room temperature. The reaction mixture was diluted with CH₂Cl₂ and plugged through a pad of celite. Purification by flash chromatography (hexanes/EtOAc 4/1) provided the amide (80 mg, 26%). ¹H NMR δ1.09 (s, 6H), 1.28-1.40 (m, 4H), 1.52 (quint, 2H, J=6.3 Hz), 1.73 (quint, 2H, J=7.4 Hz), 2.00-2.12 (m, 2H), 2.20 (t, 2H, J=7.4 Hz), 2.77 (t, 2H, J=5.5 Hz), 2.80 (t, 2H, J=5.5 Hz), 2.92 (t, 2H, J=5.5 Hz), 3.62 (dt, 2H, J=6.3, 5.5 Hz), 3.87 (s, 3H), 4.33 (d, 2H, J=5.8 Hz), 5.12-5.25 (m, 2H), 5.30-5.41 (m, 6H), 5.72 (br s, 1H), 6.74-6.87 (m, 3H). Anal. Calcd. for C₃₀H₄₅O₄N.0.6H₂O: C, 72.87; H, 9.41. Found: C, 72.77; H, 9.34.

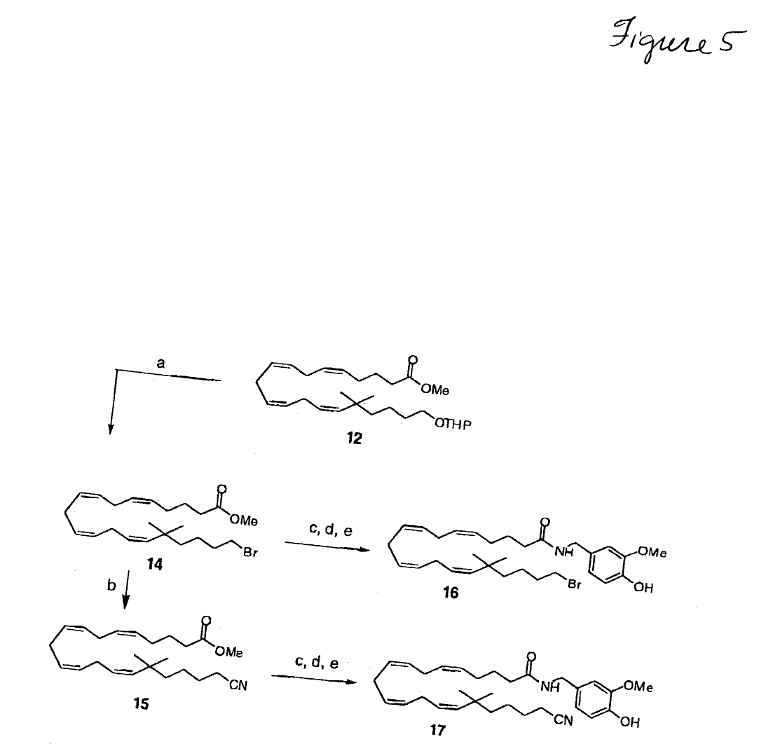

20-Bromo-16,16-dimethyl-eicosa-5,8,11,14-all-cis-tetraenoic acid methyl ester 14 (FIG. 5): To a stirred solution of triphenylphosphine (524 mg, 2 mmol) in CH₂Cl₂ (4 mL) cooled to 0° C. was added bromine (0.1 mL, 1.95 mmol). The tetraene 12 (850 mg, 1.9 mmol) (FIG. 5) was then added dropwise and the ice bath was removed. The reaction mixture was stirred overnight at room temperature. The solvent was removed under vacuum and the residue was dissolved in hexanes/EtOAc (9/1) and plugged through a pad of silica gel. Further purification by flash chromatography provided the desired bromide 14 (404 mg, 50%) (FIG. 5). ¹H NMR δ1.10 (s, 6H), 1.25-1.40 (m, 4H), 1.70 (quint, 2H, J=7.2 Hz), 1.82 (t, 2H, J=7.2 Hz), 2.31 (t, 2H, J=7.2 Hz), 2.75-2.95 (m, 6H), 3.40 (t, 2H, J=6.6 Hz), 3.66 (s, 3H), 5.12-5.41 (m, 8H).

20-Cyano-16,16-dimethyl-eicosa-5,8,11,14-all-cis-tetraenoic acid methyl ester 15: A mixture of bromide 14 (318 mg, 0.68 mmol) and KCN (90 mg, 1.36 mmol) in DMSO (3.4 mL) was heated at 50° C. for 5 hours. After cooling, the reaction mixture was diluted with hexanes/EtOAc (4/1) and plugged through a pad of silica gel. Further purification by flash chromatography afforded the cyano derivative 15 (195 mg, 78%) (FIG. 5). ¹H NMR δ 1.11 (s, 6H), 1.30-1.49 (m, 4H), 1.63 (t, 2H, J=7.6 Hz), 1.70 (quint, 2H, J=7.2 Hz), 2.00-2.12 (m, 2H), 2.32 (t, 2H, J=7.6 Hz), 2.75-2.95 (m, 6H), 3.66 (s, 3H), 5.17-5.25 (m, 2H), 5.31-5.43 (m, 6H).

20-Bromo-16,16-dimethyl-eicosa-5,8,11,14-all-cis-tetraenoic acid (4-hydroxy-3-methoxy-benzyl) amide 16 (O-1861) (FIG. 5): To a stirred solution of bromide 14 (404 mg, 0.97 mmol) (FIG. 5) in MeOH (48 mL) and water (16 mL) was added LiOH.H₂O (267 mg, 6.8 mmol) and the mixture was stirred at room temperature overnight. It was diluted with ether and acidified with 10% HCl. The layers were separated and the aqueous layer was extracted with ether. The combined organic layers were dried over MgSO₄ and evaporated to yield the crude acid (375 mg, 94%) which was used directly. The acid (200 mg, 0.49 mmol) was dissolved in CH₂Cl₂ (5 mL) and cooled to 0° C. A 2M solution of oxalyl chloride in CH₂Cl₂ (0.49 mL) was added dropwise followed by 2 drops of DMF. The ice bath was removed and the mixture was stirred at room temperature for 2 hours. The solvent was evaporated under vacuum. A solution of the acid chloride in CH₂Cl₂ (3 mL) was added to a solution of 4-hydroxy-3-methoxy benzylamine (0.4 mL, 5 mmol) in CH₂Cl₂ (5 mL) at 0° C. The ice bath was removed and the mixture was stirred at room temperature overnight. It was diluted with CH₂Cl₂ and washed with brine. The organic layer was dried over MgSO₄ and then concentrated in vacuo. The crude product was purified by flash chromatography (hexanes/EtOAc 1/1) to yield the amide 16 (85 mg, 32%) (FIG. 5). ¹H NMR δ1.10 (s, 6H), 1.31-1.45 (m, 4H), 1.73 (quint, 2H, J=7.4 Hz), 1.82 (quint, 2H, J=6.9 Hz), 2.00-2.12 (m, 2H), 2.20 (t, 2H, J=7.4 Hz), 2.77 (t, 2H, J=5.5 Hz), 2.81 (t, 2H, J=5.5 Hz), 2.91 (t, 2H, J=5.5 Hz), 3.40 (t, 2H, J=6.9 Hz), 3.86 (s, 3H), 4.34 (d, 2H, J=5.8 Hz), 5.12-5.25 (m, 2H), 5.30-5.41 (m, 6H), 5.59 (s, 1H), 5.62 (br s, 1H), 6.74-6.87 (m, 3H). Anal. Calcd. for C₃₀H₄₄O₃NBr.0.4H₂O: C, 65.06; H, 8.15. Found: C, 65.06; H, 7.97.

20-Cyano-16,16-dimethyl-eicosa-5,8,11,14-all-cis-tetraenoic acid (4-hydroxy-3-methoxy-benzyl) amide 17 (O-1895) (FIG. 5): Prepared as described for 20-Bromo-16,16-dimethyl-eicosa-5,8,11,14-all-cis-tetraenoic acid (4-hydroxy-3-methoxy-benzyl) amide 16 (28%) (FIG. 5). ¹H NMR δ1.10 (s, 6H), 1.31-1.45 (m, 4H), 1.62 (quint, 2H, J=7.1 Hz), 1.73 (quint, 2H, J=7.4 Hz), 2.00-2.12 (m, 2H), 2.20 (t, 2H, J=7.4 Hz), 2.33 (t, 2H, J=7.2), 2.77 (t, 2H, J=5.5 Hz), 2.81 (t, 2H, J=5.5 Hz), 2.91 (t, 2H, J=5.5 Hz), 3.86 (s, 3H), 4.34 (d, 2H, J=5.8 Hz), 5.12-5.25 (m, 2H), 5.30-5.41 (m, 6H), 5.59 (s, 1H), 5.62 (br s, 1H), 6.74-6.87 (m, 3H). Anal. Calcd. for C₃₁H₄₄O₃N₂.0.4H₂O: C, 75.29; H, 9.01. Found: C, 74.99; H, 8.87.

(R)-(16,16-Dimethyl-20-hydroxyeicosa-cis-5,8,11,14-tetraenoyl)-1′-hydroxy-2′-propylamine (O-1811). A mixture of tetraene 12 (200 mg, 0.45 mmol) (FIG. 5), (R)-2-aminopropanol (0.35 mL, 4.5 mmol) and NaCN (2 mg, 0.045 mmol) in MeOH (1 mL) was heated at 50° C. overnight in a sealed tube. After cooling, the mixture was diluted with CH₂Cl₂ and washed with water. The organic layer was then dried, evaporated and the residue was chromatographed to provide the amide (110 mg, 50%). ¹H NMR δ1.10 (s, 6H), 1.16 (d, 3H, J=6.9 Hz), 1.28-1.42 (m, 4H), 1.45-1.63 (m, 8H), 1.70 (quint, 2H, J=7.4 Hz), 2.03-2.12 (m, 2H), 2.18 (t, 2H), 2.18 (t, 2H, J=7.4 Hz), 2.75-2.95 (m, 6H), 3.38-3.41 (m, 1H), 3.47-3.54 (m, 2H), 3.83-3.90 (m, 1H), 4.00-4.09 (m, 1H), 4.56 (br t, 1H, J=2.5 Hz), 5.12-5.41 (m, 8H), 5.64 (br s, 1H). It was then deprotected as follows; A solution of the above amide (200 mg, 0.41 mmol) and Dowex 50Wx8-100 (300 mg) in MeOH (40 mL) was stirred at room temperature for 24 hours. The mixture was then filtered through a pad of celite and concentrated. The residue was purified by flash chromatography to provide O-1811 (120 mg, 73%). ¹HNMR δ1.10 (s, 6H), 1.16 (d, 3H, J=6.9 Hz), 1.28-1.42 (m, 4H), 1.53 (quint, 2H, J=6.3 Hz), 1.71 (quint, 2H, J=7.4 Hz), 2.00-2.12 (m, 2H), 2.19 (t, 2H, J=7.4 Hz), 2.78-2.95 (m, 6H), 3.49 (ddd, 1H, J=5.8, 9.9, 16.8 Hz), 3.61-3.69 (m, 1H), 3.63 (t, 2H, J=6.6 Hz), 4.02-4.13 (m, 1H), 5.12-5.41 (m, 8H), 5.64 (br s, 1H). Anal. (C₂₅H₄₃O₃N.0.5H₂O) Theory: C 72.42, H 10.69. Found: C 72.43, H 10.67.

(R)-(20-cyano-16,16-Dimethyleicosa-cis-5,8,11,14-tetraenoyl)-1′-hydroxy-2′-propylamine (O-1812). Prepared from 15 (FIG. 5), (R)-2-aminopropanol and NaCN in methanol as in the case of O-1811. The target compound O-1812 was obtained as an oil (79%). ¹H NMR δ1.10 (s, 6H), 1.15 (d, 3H, j=6.9 Hz), 1.28-1.49 (m, 4H), 1.56-1.65 (m, 2H), 1.71 (quint, 2H, J=7.4 Hz), 2.00-2.15 (m, 2H), 2.19 (t, 2H, J=7.2 Hz), 2.34 (t, 2H, J=6.9 Hz), 2.78-2.95 (m, 6H), 3.47-3.57 (m, 1H), 3.61-3.69 (m, 1H), 4.00-4.10 (m, 1H), 5.19-5.24 (m, 2H), 5.30-5.43 (m, 6H), 5.64 (br s, 1H). Anal. (C₂₆H₄₂O₂N_{2.}0.6H₂O) Theory: C 73.71, H 10.23. Found: C 73.54, H 10.20.

(R)-(20-Bromo-16,16-Dimethyleicosa-cis-5,8,11,14-tetraenoyl)-1′-hydroxy-2′-propylamine (O-1860). To a stirred solution of bromide 14 (404 mg, 0.97 mmol) (FIG. 5) in MeOH (48 mL) and water (16 mL) was added LiOH.H₂O (267 mg, 6.8 mmol) and the mixture was stirred at room temperature overnight. It was diluted with ether and acidified with 10% HCl. The layers were separated and the aqueous layer was extracted with ether. The combined organic layers were dried over MgSO₄ and evaporated to yield the crude acid (375 mg, 94%) which was used directly. The acid (200 mg, 0.49 mmol) was dissolved in CH₂Cl₂ (5 mL) and cooled to 0° C. A 2M solution of oxalyl chloride in CH₂Cl₂ (0.49 mL) was added dropwise followed by 2 drops of DMF. The ice bath was removed and the mixture was stirred at room temperature for 2 hours. The solvent was evaporated under vacuum. A solution of the acid chloride in CH₂Cl₂ (3 mL) was added to a solution of (R)-2-aminopropanol (0.4 mL, 5 mmol) in CH₂Cl₂ (5 mL) at 0° C. The ice bath was removed and the mixture was stirred at room temperature overnight. It was diluted with CH₂Cl₂ and washed with brine. The organic layer was dried over MgSO₄ and condensed. The crude product was purified by flash chromatography (hexane/EtOAc 1/1) to yield the amide (150 mg, 66%). ¹H NMR δ1.10 (s, 6H), 1.16 (d, 3H, J=6.9 Hz), 1.28-1.45 (m, 4H), 1.71 (quint, 2H, J=7.4 Hz), 1.83 (quint, 2H, J=6.9 Hz), 2.00-2.12 (m, 2H), 2,20 (t, 2H, J=7.4 Hz), 2.78-2.95 (m, 6H), 3.40 (t, 2H, J=6.9 Hz), 3.52 (ddd, 1H, J=4.9, 6, 11 Hz), 3.66 (ddd, 1H, J=3.3, 6, 11 Hz), 4.07 (ddq, 1H, J=3.3, 4.9, 6.9 Hz), 5.14-5.26 (m, 2H), 5.34-5.44 (m, 6H), 5.57 (br s, 1H).Anal. (C₂₅H₄₂O₂NBr.0.25HCCl₃) Theory: C 60.24, H 8.54. Found: C 60.24, H 8.54

(R)-4-morpholin-4-yl-butyric acid 2-eicosa-5,8,11,14-tetraenoylamino-propyl ester (O-2140). Arachidonic acid (542 μL, 1.64 mmol) was dissolved in benzene (12 mL) and 2 drops of DMF. Oxalyl chloride (286 μL, 3.28 mmol) was added dropwise at 0° C. On complete addition the ice-bath was removed and the mixture was stirred at 25° C. for one hour. Re-cooled the mixture to 0° C. and added a solution of (R)-(−)-2-amino-1-propanol (1.23 g, 16.4 mmol) in THF (12 mL). The ice-bath was removed and the mixture was stirred at 25° C. for 20 minutes. It was diluted with chloroform and washed with 10% HCl and 10% NaOH solutions, and dried (MgSO₄). The crude product was purified by flash chromatography (5% MeOH/CHCl₃; R_{f} 0.3) to yield the amide (580 mg, 98%). ¹H NMR δ0.89 (t, 3H, 7.1 Hz), 1.17 (d, 3H, 6.9 Hz), 1.18-1.42 (m, 6H), 1.69-1.78 (m, 2H), 2.01-2.22 (m, 6H), 2.76-2.94 (m, 6H), 3.45-3.57 (m, 1H), 3.62-3.71 (m, 1H), 4.01-4.19 (m, 1H), 5.28-5.49 (m, 8H), 5.62 (br.d, 1H, 7.9 Hz). The amide (300 mg, 0.83 mmol) was dissolved in CH₂Cl₂ (21 mL) with DCC (273 mg, 1.29 mmol) and 4-morpholin-4-yl-butyric acid (243 mg, 1.16 mmol). The suspension was stirred overnight at 25° C. Diluted with CH₂Cl₂, washed with saturated NaHCO₃, brine, and dried (MgSO₄). Purification by flash chromatography (15% MeOH/CHCl₃; R_{f} 0.85) gave 180 mg (42%) of the amide. ¹H NMR δ0.89 (t, 3H, 6.9 Hz), 1.16 (d, 3H, 6.9 Hz), 1.22-1.45 (m, 6H), 1.64 (q, 2H, 7.4 Hz), 1.81 (q, 2H, 6.8 Hz), 2.01-2.22 (m, 6H), 2.31-2.49 (m, 8H), 2.77-2.86 (m, 6H), 3.69 (t, 4H, 4.7 Hz), 4.02 (dd, 1H, 4.4, 14.7 Hz), 4.11 (dd, 1H, 4.4, 14.7 Hz), 4.22-4.31 (m, 1H), 5.28-5.49 (m, 8H), 5.53 (br.d, 1H, 7.9 Hz). To a solution of amide (154 mg, 0.3 mmol) in diethyl ether (6 mL), 1M HCl.Et₂O (450 μL) was added and the mixture was stirred at 25° C., followed by removal of solvent to yield 161 mg (97%) of the salt (1). ¹H NMR (d₆-DMSO) δ0.86 (t, 3H, 6.3 Hz), 1.03 (d, 3H, 6.6 Hz), 1.21-1.39 (m, 6H), 1.54 (q, 2H, 7.4 Hz), 1.92 (q, 2H, 6.8 Hz), 1.99-2.11 (m, 6H), 2.42 (t, 2H, 6.8 Hz), 2.73-2.85 (m, 6H), 2.95-3.11 (br.m, 4H), 3.30-3.45 (br.m, 2H), 3.71-4.05 (m, 7H), 5.26-5.41 (m, 8H), 7.81 (br.d, 1H, 7.9 Hz). Anal. Calcd for C₃₁H₅₃N₂O₄Cl.0.4 CHCl₃: C, 62.76; H, 8.96; N, 4.66. Found: C, 62.12; H, 8.86; N, 4.62.

### EXAMPLE 2

A study was conducted which was aimed at developing new and metabolically stable AEA and arvanil analogs that would exhibit (i) high selectivity for the CB₁ cannabinoid receptor versus the CB₂ or VR₁receptors, or (ii) high potency at both CB₁ and VR₁receptors. Selective CB₁ or VR₁agonists can be exploited as tools for in vivo pharmacological studies on AEA. By comparing qualitatively and quantitatively the pharmacological profiles of AEA and these selective antagonists, it is possible to study the relative involvement of CB₁ or VR₁ receptors in the pharmacological actions of AEA. CB₁/VR₁ “hybrid” agonists with similar potency at these two receptor classes could be used themselves or as templates for the development of ultra-potent analgesic, vasodilator and anti-tumor agents.

In order to develop new selective compounds or “hybrid” agonists, the inventors chose to modify the chemical structures of two AEA derivatives that had been previously shown to be more resistant to AEA to enzymatic hydrolysis and to activate both CB₁ and VR₁ receptors. Met-AEA is at least approximately 100 fold more potent at CB₁ than VR₁ receptors, and arvanil is approximately 500 fold more potent at VR₁ than CB₁ receptors.

### MATERIALS AND METHODS

Over-expression of hVR₁ CDNA into HEK 293 cells was carried out as described in Hayes et al., Pain 88:205-215 (2000). HEK-hVR₁ cells were grown as monolayers in minimum essential medium supplemented with non-essential amino acids, 10% fetal calf serum and 0.2 mM glutamine, and maintained under 95%/5% O₂/CO₂ at 37° C. N18TG2 and RBL-2H3 cells were grown as described in Melck et al, Bioch. Biophys. Res. Commun. 262:275-284 (1999). Ionomycin was purchased from Sigma. DMH-arvanil, and six substituted dimethylpentyl (DMP) derivatives of arvanil and met-AEA were synthesized as described in Example 1.

For CB₁ receptor binding, [³H]CP-55,940 (Kd=690 nM) was incubated with P₂ membranes from whole rat brains as described in Compton et al., J. Pharm. Exp. Ther. 265:218-226 (1993). Displacement curves were generated by incubating drugs with 1 nM of [³H]CP-55,940. The assays were performed in triplicate, and the results represent the combined data from three individual experiments. A separate set of experiments were conducted in which phenylmthylsulfonylfluoride (PMSF) was added at a concentration of 100 μm in order to prevent possible metabolism of the analogs. CB₂ binding assays were performed as described in Melek et al., ibid. In all cases, Kᵢ values were calculated applying the Cheng-Prusoff equation to the IC₅₀ values (obtained by GraphPad) for the displacement of the bound radioligand by increasing concentrations of the test compounds.

The effect of the substances on the influx of Ca²⁺ into cells was determined by using Fluo-3 methylester (Molecular Probes), a selective intracellular fluorescent probe for Ca²⁺. HEK-hVR₁ cells were prepared and loaded as described in De Petrocellis, et al., FEBS Lett. 483:52-56 (2000). Experiments were carried out by measuring fluorescence at 25° C. (λ_{EX}=488 nm, λ_{EM}=540 nm) before and after the addition of test compounds at various concentrations. Capsazepine (1-5 μM) was added 30 minu. Before the test compounds. Data are expressed as the concentration exerting a half-maximal effect (EC₅₀) calculated by using GraphPad software. The efficacy of the effect was determined by comparing it to the analogous effect observed for 4 μM ionomycin.

The effect of compounds on the enzymatic hydrolysis of [¹⁴C] AEA (6 μM) was studied by using membranes prepared from N18TG2 cells incubated with increasing concentrations of compounds in 50 mM Tris-HCL, pH 9, for 30 min at 37° C. [¹⁴C] Ethanolamine produced from [¹⁴C] AEA hydrolysis was quantified by scintillation spectroscopy and used to measure fatty acid amide hydrolase (FAAH) activity. The effect of compounds on the uptake of AEA by RBL-2H3 cells was studied with a procedure analogous to that described in Melck, ibid., and modified as described in Hilliard et al., J. Neurochem. 69:631-638 (1997). Cells were incubated with [¹⁴C] AEA (4μM) for 5 min. at 37° C., in the presence or absence of varying concentrations of the inhibitors. Residual [1⁴C] AEA in the incubation media after extraction with CHCl₃/CH₃0H (2:1 by volume) was used as a measure of the AEA that was taken up by cells. Data were expressed as the concentration exerting 50% inhibition of AEA hydrolysis and uptake (IC₅₀), calculated with GraphPad.

For pharmacological assays in vivo, cannabinoids were dissolved in a 1:1:18 mixture of ethanol, emulphor, and saline for i.v. administration. Mice received the analog by tail-vein injection and were evaluated for their ability to produce hypomotility, hypthermia, immobility and antinociception. These parameters were determined using a slight modification to the approach described in Compton, ibid., consisting of a shorter testing time and of only two measures being made in the same animal. In the first group of animals, antinociception was determined using the tail-flick reaction time to a heat stimulus. Before vehicle or drug administration, the baseline latency period (2-3 s) was determined. Four minutes after the injection, tail-flick latency was assessed once more, and the differences in control and test latencies was calculated. A 10-s maximum latency was used to calculate % maximal possible effect (MPE). These animals were then transferred immediately to individual photocell activity chambers (11 inches×6.5 inches), and spontaneous activity was measured during the next 10 min period. The number of interruptions of 16 photocell beams per chamber was recorded, and the activity in the drug treated groups was expressed as a percentage of the vehicle-treated animals. In a separate group of mice, rectal temperature was determined prior to vehicle or drug administration with a telethermometer (Yellow Springs Instrument Co., Yellow Springs, Ohio) and a thermistor probe (model YSI 400, Markson, Inc.) inserted at a depth of 2 cm. At 4 min after i.v. injection of drug or vehicle, rectal temperature was measured again, and the difference between pre- and post-injection values was calculated. These animals were then placed on a metal ring (5.5 cm in diameter) that was attached to a stand at a height of 16 cm. The amount of time (s) that the mouse spent motionless during a 5-min test session was recorded. The criterion for immobility was the absence of all voluntary movements (excluding respiration, but including whisker movements). The immobility index was calculated as described in Compton, ibid.

### RESULTS

Table 1 shows the affinity of the tested AEA derivatives for CB₁ and CB₂ Cannabionid receptors, potency, and efficacy at Human VR₁receptors and the effect on the anandamide membrane transporter (AMT) in intact RBL-2-H3 cells and fatty acid amide hydrolase (FAAH) in N18TG2 Cells.

TABLE 1

| | | | VR₁ | | AMT | FAAH |
|---|---|---|---|---|---|---|
| | CB₁ | CB₂ | (EC₅₀, | VR₁ | (IC₅₀, | (IC₅₀, |
| Analogs | (Kᵢ, nM) | (Kᵢ, nM) | nM) | efficacy | μM) | μM) |
| 0-1811 | 115.2 ± 25.2 | 800.1 ± 150.2 | 724 ± 99 | 62.5 ± 0.8 | 42.5 ± 3.3 | >50 |
| | 116.1 ± 11.1* | | | | | |
| 0-1812 | 3.4 ± 0.5 | 3870 ± 235 | 1949 ± 184 | 50.0 ± 1.6 | 37.0 ± 4.5 | >50 |
| | 4.6 ± 0.4* | | | | | |
| 0-1860 | 2.2 ± 0.2 | >10000 | 371 ± 34 | 62.1 ± 5.7 | 40.0 ± 4.5 | >50 |
| | 1.0 ± 0.1* | | | | | |
| 0-1839 | 261.8 ± 90.2 | >10000 | 0.7 ± 0.08 | 71.9 ± 4.5 | 19.3 ± 2.3 | >50 |
| | 201.0 ± 4.1* | | | | | |
| 0-1856 | 789.7 ± 105.4 | >10000 | 5.0 ± 0.7 | 83.9 ± 5.1 | 40.0 ± 3.8 | 14.5 ± 3.4 |
| | 1070 ± 134* | | | | | |
| 0-1895 | 67.0 ± 7.5 | 5000 ± 850 | 1.0 ± 0.2 | 83.1 ± 6.1 | 38.4 ± 3.1 | 18.0 ± 2.7 |
| | 44.8 ± 8.3* | | | | | |
| 0-1861 | 32.6 ± 1.2 | >10000 | 30.0 ± 0.2 | 74.0 ± 4.5 | 13.5 ± 1.9 | >50 |
| | 24.3 ± 2.6* | | | | | |

In Table 1, affinity for CB₁ and CB₂ receptors was measured in displacement assays carried out using rat brain or spleen membranes and [³H]CP 55,940 or [³H]WIN44,212-2, respectively. Potency at human VR₁ was established by measuring the stimulatory activity of the compounds on the Ca²⁺ intracellular concentration in HEK cells transfected with cDNA encoding the human VR₁ receptor. Efficacy at VR₁ was measured by expressing the effect of a maximal concentration of each compound (usually 10 μM) as percent of the maximal possible effect obtained with 4 μM ionomycin. Data are means ±SEM of at least n=3 separate determinations. *Kᵢ was determined in the presence of PMSF.

As can be seen from Table 1, the compounds all inhibited the binding of [³H] CP55,940 to rat brain membranes, although with different Kᵢ values. The analogs with the highest and lowest affinities were the bromo derivative of DMP-Met-AEA (O-1860, Kᵢ 2.2 nM) and the hydroxyl derivative of DMP-arvanil (O-1856, Kᵢ 790 nM), respectively. Within each series, the presence of a cyano and a bromine function on the C-20 yielded the highest affinity for the CB₁ receptor, while the hydroxyl analog produced the lowest affinity. In fact, the affinities of the DMP-bromo (O-1861) and the DMP-cyano (O-1895) derivatives were 3- to 6-fold greater than that of the corresponding DMH-derivative of arvanil, O-1839 (Kᵢ 262 nM). However, the bromo (O-1860) and cyano (O-1812) derivatives in the DMB-Met-AEA series had only slightly greater CB₁ affinity than DMH-Met-AEA (Kᵢ 7.0 nM (see Ryan et al., J. Med. Chem.40:3617-3625 (1997)). Finally, the N-(3-methoxy-4-hydroxy-phenyl)-group instead of the (R)-1′-methyl-2′-hydroxy-ethyl “head” in AEA also reduces affinity, since the arvanil-like compounds were 7-20 fold less potent than the corresponding Met-AEA-like compounds.

The compounds were also assayed in the presence of the FAAH inhibitor PMSF in order to assess whether inhibition of FAAH influenced their CB₁ affinity. The Kᵢ values observed for the compounds under these conditions did not greatly differ from those that measured thin the absence of PMSF.

High micromolar concentrations of almost all compounds were needed to displace [³H]WIN55,212-2 from it binding sites in rat spleen membranes, which contain mostly CB₂ receptors. The generally very low affinity of these compounds for the CB₂ receptors demonstrates that these compounds are selective CB₁ ligands and that the cyano and bromo analogs in both series are highly selective CB₁ probes. The hydroxy analog is much less CB₁ vs. CB₂ selective in both series.

Table 1 shows that all the compounds tested induced a VR₁-mediated increase of cytosolic Ca²⁺ concentration in HEK cells over-expressing the human VR₁ receptor. The effect was not observed in the presence of the VR₁ antagonist, capsazepine (5 μM, data not shown). The most potent (EC₅₀ 0.7-5.0 nM) and efficacious (maximum effect 71.9-83.9% of the effect observed with 4 μm ionomycin) compounds were those belonging to the arvanil series, which were at least two orders of magnitude more potent than the compounds in the Met-AEA series (EC₅₀ 371-1950 nM, maximum effect 50.0-62.5%). Within each series, the presence of bromine, cyanide, or hydroxy groups on the C-20 did not cause the same effect on activity. In fact, in the arvanil series, the most potent compound was O-1839 and the least potent was )-1856, i.e., the DMH- and 20-hydroxy DMP-arvanil derivatives, respectively, whereas in the Met-AEA series the most potent compound was O-1860 (EC₅₀ 0.37 μM, maximum effect 50.0%) and the least potent was O-1812 (1.9 μM, maximum effect 50.0%), i.e., the 20-bromo and 20-cyano derivatives of DMP-Met-AEA, respectively. These data suggest that the structural pre-requisites regulating the efficacy and potency of AEA analogs for VR₁ receptors are different from, although in some cases overlapping with, those necessary for interaction with CB₁ receptors.

Overall, these findings suggest (i) that the presence of an N-Vanillyl (3-methoxy-4-hydroxy-phenyl) moiety is necessary for very high potency at vanilloid receptors, (ii) that addition of two methyl groups on the C-16 and elongation of the terminal aliphatic chain of arvanil are not detrimental to its vanilloid activity, and (iii) addition of either a bromo or a cyano group on the C-20 of AEA analogs can be exploited to increase their potency at CB₁ receptors. A corollary to these observations is that O-1812 and O-1860 are the first CB₁ ligands to be developed that are truly selective versus VR₁ receptors. In particular, O-1812 is 580 fold and 1000 fold more potent on CB₁ than on VR₁ or CB₂ receptors, respectively. The addition of two methyl groups on the C-16 and either a bromine or a cyano group on the C-20 increased the affinity of arvanil analogs for CB₁ receptors without appreciably altering its activity at VR₁ receptors. The resulting compounds, i.e., O-161 and O-1895, are 10 to 60-fold more potent at VR₁ than at CB₁ receptors, and represent tru “hybrid” agonists that may be advanatageously exploited as multitarget thereapeutic agents.

In consideration of the potent inhibition of the AMT previously observed with arvanil and its derivatives (see Melck, ibid), the seven compounds were also tested for their inhibitory effect on [⁴C] AEA uptake by RBL-2H3 cells, where the AMT transporter has been previously characterized (see Table 1 above). All the arvanil derivatives tested were at least four fold less potent than arvanil (whose IC₅₀ is 3.6 μM), the most potent one being O-1861 (IC₅₀ 13.5 μM) and the least potent O-1856 (42 μM). Under the same conditions, the AMT inhibitor AM404 exhibits an IC₅₀ of 8.1 μM (data not shown). Thus, the results in Table 1 confirm the existence of a partial overlap between the ligand recognition properties of VR₁ receptors and the AMT (14.24). In fact, the compounds with the highest potency at VR₁ receptors (O-1839, O-1861, and O-1895) were also active on the AMT, and, conversely, the two least potent VR₁ agonists (O-1811 and O-1812) were also very weak AMT inhibitors. However, since even the most potent AMT inhibitors tested were less potent than arvanil, it can be suggested that branching and either elongation of the alkyl chain or introduction of hindering electro-negative groups on the C-20 reduces the affinity of AEA analogs for the AMT. In support of this, it was also found that the Met-AEA analogs tested were less potent AMT inhibitors than MET-AEA (IC₅₀ 24.5 μM (see Rakhshan, J. Pharmacol. Exp. Ther. 292:960-967 (2000) wherein RBL-2H3 cells are used under conditions similar to those described herein).

The finding that the affinity of all the tested compounds for CB₁ receptors was not increased in the presence of a FAAH inhibitor indirectly suggests that these compounds are not hydrolyzed to a significant extent during the incubation with rat brain membranes and, hence, are not good substrates for FAAH. Accordingly, when the compounds were tested for their capability of inhibiting [¹⁴C] AEA hydrolysis by FAAH-containing N18TG2 cell membrane preparations, they were mostly inactive (see Table 1). This is in agreement with previous observations that Me-AEA, DMH-derivatives of AEA and arvanil are poor substrates for FAAH. The two exceptions were O-1856 and O-1895, which did inhibit the hydrolysis of [¹⁴C] AEA, although not very potently. It is possible that on the C-20 of AEA analogs, the presence of either a hydroxy or a cyanide group, both capably of exchanging protons within its active site of the enzyme, confers to these compounds the ability to interact with FAAH and subsequently inhibit the enzyme, as previously suggested for the “head” group of arachidonoyl-seritonin (see Bisogno et al., Biochem. Biophys. Res. Commun. 248:515-522 (1998)).

It was also assessed whether a correlation exists between the potency in vivo of the anandamide and arvanil analog compounds set forth in Table 1 and their affinity/efficacy at CB₁ and VR₁ receptors. Table 2 shows the effects of the anandamide and arvanil analogs in depressing spontaneous activity (S.A.) and rectal temperature (R.T.) and increasing tail-flick (T.F.) response and relative immobility (R.I.) or catalepsy in mice following i.v. administration.

TABLE 2

| Analogs | S.A. | T.F. | R.T. | R.I. | Average |
|---|---|---|---|---|---|
| O-1811 | 0.996 | 1.243 | 0.188 | 0.663 | 0.773 |
| O-1812 | 0.017 | 0.014 | 0.050 | 0.017 | 0.025 |
| O-1860 | 0.390 | 0.290 | 0.440 | 0.430 | 0.388 |
| O-1839 | 0.080 | 0.074 | 0.080 | N.D. | 0.078 |
| O-1856 | 0.470 | 0.810 | 1.450 | 0.760 | 0.873 |
| O-1895 | 0.265 | 0.263 | 0.179 | 0.168 | 0.219 |
| O-1861 | 0.060 | 0.104 | 0.053 | 0.055 | 0.068 |

The results in Table 2 are presented as ED₅₀ and are expressed in mg/kg. The average ED₅₀ for each compound to produce the four effects is in the right hand column.

All of the compounds were potent in depressing spontaneous activity and rectal temperature and in producing analgesia and catalepsy in mice following i.v. administration. Additionally, none of the compounds exhibited any appreciable selectivity in producing one effect versus the others. The only exception was O-1811, which was approximately 10-fold more potent in reducing body temperature than in increasing tail-flick latency. The average ED₅₀ values for the four pharmacological effects for each compound are also presented in Table 2. The individual ED₅₀ values along with these averages clearly demonstrate the compounds varied significantly from each other with regard to potency. Two of the most potent analogs were the cyano derivative in the Met-AEA series (O-1812) and the bromo derivative in the arvanil series (O-1861). Interestingly, the corresponding derivatives in the opposite series, that is the cyano derivative in the arvanil series (O-1895) and the bromo analog in the Met-AEA series (O-1860) were 5-10 times less potent. The potency of DMH-arvanil (O-1839) was similar to that of the O-1861 indicating than an ethyl and bromo terminal group influence potency to the same degree. The hydroxy analogs in both series (O-1811 and O-1856) were the least potent of all analogs.

These data provide insights in the molecular mode of action of the compounds in vivo. If the actions of the compounds in the mouse ‘tetrad’ of neurobehavioral tests were uniquely mediated by CB₁ receptors, there should have been observed a finding that O-1860 and O-1812 were the most potent in the four tests. Conversely, if VR₁ receptors had played a major role in the neurobehavioral effects of the compounds, there should have been observed high potency for O-1839 and O-1856. Finally, if both receptors were equally and synergistically involved in these in vivo actions of AEA analogs, it should have been found that the highest potency was with O-1861 and O-1895.

It was found, that, indeed, the most potent VR₁ agonist in the study was O-1839. O-1839 was very potent in the ‘tetrad’ of tests, but the second most potent VR₁ agonist, O-1895, was considerably less potent. Furthermore, of the two most potent and selective CB₁ ligands tested, only 1812 was extremely potent in the mouse ‘tetrad’ of tests. Finally, between the two “hybrid” CB₁/VR₁ agonists described herein, significantly different potencies in vivo were observed. Overall, although almost all compounds examiner here were more potent than AEA both in these in vivo tests and as either CB₁ ligands or VR₁ agonists, no correlation could be found between their ED₅₀ values in the ‘tetrad’ and their K₁ values as CB₁ ligands or their ED₅₀ values as VR₁ agonists. Even within each of the two classes of compounds examined here, the Met-AEA and arvanil derivatives (“analogs”), little correlation can be found between potencies at receptors and in vivo. These observations reinforce previous findings that suggest that some effects of arvanil and AEA in mice are not confined to CB₁ and VR₁ receptors. With the exception of O-1812, whose high potency in vivo may still be due mostly to CB₁ receptor activation, and with the inclusion of arvanil, whose ED₅₀ in the ‘tetrad’ is 0.085 mg/kg, the only compounds examined here that exhibit average ED₅₀ <0.1 mg/kg in vivo have in common the vanilloid moiety, which may thus be an important, although not sufficient, structural determinant not only for VR₁ activation but also for recognition of the putative non-CB₁, non-CB₂, non-VR₁ sites of action. An alternative explanation could be that this structural feature confers to AEA analogs pharmacokinetic properties that allow them to reach CB₁ or VR₁ receptors more efficaceously. However, this premise is not in agreement with the much lower potency in vivo of O-1895 and O-1856. For these two latter compounds, in fact, the high potency at either CB₁ or VR₁ receptors, and in the presence of an N-vanillyl moiety (were this to improve pharmacokinetic properties), should have led to high potency in vivo.

None of the structural features of the compounds analyzed here, e.g., the presence of branching on the C-16; of cyanide, bromo and hydroxyl groups on the C-20; and of a 1′-methyl-2′-hydroxy-ethyl or an N-vanillyl group instead of the ethanolamine moiety, seem to be sufficient alone to confer very high potency in the mouse ‘tetrad’ of tests. In fact, the hydroxyl group was actually detrimental to both VR₁/CB₁ activity in vitro and neurobehavioral activity.

The experiments and results in this Example describe the activity of metabolically stable VR₁ and/or CB₁ receptor agonists. In addition various aspects of the structure-activity relationships of AEA analogs for either receptor subtype as well as for the AMT and FAAH has been elucidated. These results provide evidence that some of the neurobehavioural actions of AEA and, particularly, arvanil derivatives (anandamide and arvanil analogs) are due to the interaction with novel binding sites. The compounds can be used as selective probes for biochemical studies on CB₁ or VR₁ receptors, or as novel analgesic, anti-inflammatory, vasodilator, and anti-proliferative drugs, or as templates for the same.

### EXAMPLE 3

Experiments were conducted to assess the impact of chemical modification of the amide and aromatic moieties of arvanil, particularly as to whether such modifications lead to arvanil analogs which are CB₁/VR₁ hybrid activators with cannabimimetic activity. Therefore, the activity of eight novel compounds, obtained from arvanil by modifying these two regions, was analyzed here on: 1) CB₁ and VR₁ receptors; 2) the AMT; and 3) the fatty acid amide hydrolase (FAAH). The latter enzyme is responsible for AEA hydrolysis (See, Cravatt et al., Nature 384:83-87 (1996), and Ueda et al., Chem. Phys. Lipids 108:107-121 (2000) and drives in part the activity of the AMT in intact cells. Of the compounds tested, there are four potent VR₁ agonists that were 450-19,000-fold selective over CB₁ receptors. When tested in the mouse ‘tetrad’, these four compounds exhibited potent cannabimimetic activity, based on a positive response in all four tests of the ‘tetrad’. Additionally, one compound inactive at both CB₁ and VR₁ receptors was very potent in the in vivo mouse model. The results also indicate that non-CB₁ receptors are important in determining high activity in the mouse “tetrad” tests.

### MATERIALS AND METHODS

Synthesis and chemicals—Arachidonyl analogs O-1986, O-1988, O-2094 were synthesized by treatment of the appropriate amines with the acid chloride of arachidonic acid as described in Example 1. The amines used for O-1988 and O-2094 were prepared by reductive amination procedures (see, Abdel-Magid et al., J. Org. Chem.61:3849-3862 (1996)) using 3-methoxy-4-hydroxybenzaldehyde/CH₃NH₂.HCl/methanol/NaCNBH₄ for the former and 3-chloro-4-hydroxybenzaldehyde/ammonium acetate/NaCNBH₄/methanol/mol.sieves 3 A° for the latter. The urea analog O-1987 was prepared from arachidonic acid, in a one-pot reaction, via its isocynate followed by treatment with 4-hydroxy-3-methoxybenzylamine HCl (see for example, the procedures of Ng, et al., J. Med. Chem. 42:1975-1981 (1999)). The thiourea O-2095 was synthesized by treatment of vanillyl isothiocynate with norarachidonylamine (synthesized from arachidonyl isocyanate and 2-trimethylsilylethanol/80° C./16h/followed by deprotection with CF₃COOH at 0° C.). Similarly the thiourea O-2109 was prepared using 3-chloro-4-hydroxybenzylisothiocyanate (prepared from 3-chloro-4-hydroxybenzylamine using the same procedure as described for vanillyl isothiocyanate). O-2142 was synthesized from arvanil and 4-morpholino-butyric acid in methylene chloride/DCC (see, for example, the procedure of Razdan et al., J. Med. Chem. 19:454-461 (1975)). All compounds were characterized on the basis of their [¹H] nuclear magnetic resonance spectra (run on a Jeol Eclipse 300 MHz) and elemental analyses.

Guanosine-5′-O-(3-[³⁵S]thiotriphosphate) ([³⁵S]GTPγS) was purchased from Perkin Elmer Life Sciences (Boston, Mass.). [³H]CP55940 was purchased from Perkin Elmer Life Sciences (Boston, Mass.). GDP and GTPγS were purchased from Roche Molecular Biochemicals (Somerville, N.J.). All other reagent grade chemicals and enzymes were obtained from Sigma Chemical Co. (St. Louis, Mo.) or Fisher Scientific (Pittsburgh, Pa.).

Agonist-stimulated [³⁵S]GTPγS binding assays—The hippocampus of young adult rats, dissected on ice, was used for these assays since this brain area exhibits a more efficacious coupling of CB₁ receptors to G-proteins than whole brain. Each hippocampus was homogenized with a Tissumizer (Tekmar, Cincinnati, Ohio) in cold membrane buffer (50 mM Tris-HCl pH 7.4, 3 mM MgCl₂, 0.2 mM EGTA, 100 mM NaCl, pH 7.7) and centrifuged at 42,000 g for 20 min at 4° C. Pellets were re-suspended in membrane buffer, then centrifuged again at 42,000 g for 20 min at 4° C. Pellets from the second centrifugation were homogenized in membrane buffer and stored at −80° C. Frozen membranes were thawed and diluted in membrane buffer, homogenized, and preincubated for 10 min at 30° C. in 0.004 units/ml adenosine deaminase (240 units/mg protein, Sigma Chemical Co.) to remove endogenous adenosine, then assayed for protein content before addition to assay tubes. Assays were conducted at 30° C. for 1 hr in membrane buffer including 10 μg membrane protein with 0.1% (w/v) bovine serum albumin (BSA), 10 μM GDP and 0.1 nM [³⁵S]GTPγS in a final volume of 0.5 ml. Non-specific binding was determined in the absence of agonists and the presence of 30 μM unlabeled GTPγS. Reactions were terminated by rapid filtration under vacuum through Whatman GF/B glass fiber filters, followed by three washes with cold Tris-HCl buffer, pH 7.4. Bound radioactivity was determined by liquid scintillation spectrophotometry at 95% efficiency for [35S] in 4 ml BudgetSolve scintillation fluid (RPI Corp., Mount Prospect, Ill.). Net agonist-stimulated [³⁵S]GTPγS binding values were calculated by subtracting basal binding values (obtained in the absence of agonist) from agonist-stimulated values (obtained in the presence of agonist). Data analyses (including agonist concentration-effect and competition curves) were conducted by iterative non-linear regression using Prism for Windows (GraphPad Software, San Diego, Calif.) to obtain EC₅₀, Eₘₐₓ and Kᵢ values. Significant stimulation of [³⁵S]GTPγS binding was determined by ANOVA followed by Dunnett's test at the p<0.05 level to compare each concentration of ligand to basal binding. Data are expressed as means ±S.E. of experiments performed in triplicate in membranes from at least 3 different hippocampi.

CB₁ receptor binding assays—All experiments were performed with whole brain membranes rather than hippocampal membranes, and preparation of these membranes was the same as for the hippocampus. Binding was initiated by the addition of 75 μg whole brain membranes to siliconized tubes containing [³H]CP55940 (1 nM), competing ligand (concentrations from 0.001-30 μM), 0.5% (w/v) BSA and a sufficient volume of buffer (membrane buffer minus sodium chloride) to bring the total volume to 0.5 ml. The addition of 2 μM of unlabeled CP 55940 was used to assess non-specific binding. Membranes were then incubated at 30° C. for 60 minutes. The reaction was terminated by addition of ice-cold wash buffer (50 mM Tris HCl, 0.5% BSA; pH 7.4) followed by rapid filtration under vacuum through Whatman GF/B glass-fiber filters using a 96 well harvester (Brandell, Gaithersburg, Md.). The tubes were washed twice with 2 ml of ice-cold wash buffer and the filters rinsed twice with 4 ml of wash buffer. Filters were placed into 7 ml plastic scintillation vials and 5 ml BudgetSolve scintillation fluid added. Bound radioactivity was determined by liquid scintillation spectrophotometry at 45% efficiency for [³H].

Cytosolic calcium concentration ([Ca^{2+]}ᵢ) assay-Over-expression of human VR₁ cDNA into human embryonic kidney (HEK) 293 cells was carried out as described in Hayes et al., Pain 88:205-215 (2000). Cells were grown as monolayers in minimum essential medium supplemented with non-essential amino acids, 10% fetal calf serum and 0.2 mM glutamine, and maintained under 95%/5% O₂/CO₂ at 37° C. The effect of the substances on [Ca^{2+]}ᵢ was determined by using Fluo-3, a selective intracellular fluorescent probe for Ca²⁺. One day prior to experiments cells were transferred into six-well dishes coated with Poly-L-lysine (Sigma) and grown in the culture medium mentioned above. On the day of the experiment the cells (50-60,000 per well) were loaded for 2 h at 25° C. with 4 μM Fluo-3 methylester (Molecular Probes) in DMSO containing 0.04% Pluoronic. After the loading, cells were washed with Tyrode pH=7.4, trypsinized, resuspended in Tyrode and transferred to the cuvette of the fluorescence detector (Perkin-Elmer LS50B) under continuous sirring. Experiments were carried out by measuring cell fluorescence at 25° C. (λ_{EX}=488 nm, λ_{EM}=540 nm) before and after the addition of the test compounds at various concentrations. Data are expressed as the concentration exerting a half-maximal effect (EC₅₀). The efficacy of the effect was determined by comparing it to the analogous effect observed with 4 μM ionomycin.

Anandamide membrane transport assay—The effect of compounds on the uptake of [¹⁴C]AEA by rat basophilic leukemia (RBL-2H3) cells was studied by using 3.6 μM (10,000 cpm) of [¹⁴C] AEA. Cells were incubated with [¹⁴C] AEA for 5 min at 37° C., in the presence or absence of varying concentrations of the inhibitors. Residual [¹⁴C] AEA in the incubation media after extraction with CHCl₃/CH₃OH 2:1 (by vol.), determined by scintillation counting of the lyophilized organic phase, was used as a measure of the AEA that was taken up by cells. Previous studies (Bisogno et al., ibid) had shown that after a 5 min incubation the amount of [¹⁴C] AEA disappeared from RBL-2H3 cell medium is found mostly (>90%) as unmetabolized [¹⁴C] AEA inside the cells. Data are expressed as the concentration exerting 50% inhibition of AEA uptake (IC₅₀) calculated by GraphPad.

Fatty acid amide hydrolase assay—The effect of compounds on the enzymatic hydrolysis of AEA was studied using membranes prepared from frozen brains of CD rats (Charles River, France), incubated with the test compounds and [¹⁴C] AEA (9 μM) in 50 mM Tris-HCl, pH 9, for 30 min at 37° C. [¹⁴C] Ethanolamine produced from [¹⁴C] AEA hydrolysis was measured by scintillation counting of the aqueous phase after extraction of the incubation mixture with 2 volumes of CHCl₃/CH₃OH 2:1 (by vol.). Data are expressed as the concentration exerting 50% inhibition of AEA uptake (IC₅₀), calculated by GraphPad.

Pharmacological effects in mice—Cannabinoids were dissolved in a 1:1:18 mixture of ethanol, emulphor (North American Chemicals, Cranbury, N.J.) and saline for i.v. administration. The analogs were administered to mice by tail-vein injection and evaluated for their ability to produce hypomotility, hypothermia, and antinociception. These pharmacological measures were determined in the same mouse at a time when maximal activity was present. Similar to that described in Example 1, in order to measure locomotor activity, mice were placed into individual photocell activity chambers (11 inches×6.5 inches) 5 min after injection. Spontaneous activity was measured during the next 10-min period, and the number of interruptions of 16 photocell beams per chamber was recorded. Antinociception was determined using the tail-flick reaction time to a heat stimulus. Before vehicle or drug administration, the baseline latency period (2-3 sec) was determined. Twenty min after the injection tail-flick latency was assessed once more, and the differences in control and test latencies were calculated. A 10-sec maximum latency was used. Antinociception was expressed as % MPE as described below. As for hypothermia, rectal temperature was determined prior to vehicle or drug administration with a telethermometer (Yellow Springs Instrument CO., Yellow Springs, Ohio) and a thermistor probe (model YSI 400, Markson, Inc.) inserted at a depth of 2 mm. At 30 min after the injection, rectal temperature was measured again, and the difference between pre- and post-injection values was calculated.

Data Analysis—For production of hypomotility and hypothermia, the data were expressed as a percentage of control activity and change in temperature, respectively. Antinociception was calculated according to the following equation.

% MPE=−((test latency-control latency)/(10s-control latency))×100

At least six animals were treated with each dose-response relationships could be determined for each analog. ED₅₀ values were determined from least-squares unweighted linear regression analysis of log dose-response plots. Maximal effects for all compounds combined on spontaneous activity, temperature, antinociception, and catalepsy were, respectively, 90% inhibition, −5° C., 100% MPE, and 60% immobility. Thus, the ED₅₀ values indicate response levels of 45% inhibition, −2.5° C., 50% MPE, and 30% immobility.

### RESULTS

Table 3 presents test data for the arvanil analogs at CB₁ receptors, VR₁ receptors, AEA transport, and FAAH. Affinity for CB₁ receptors was measured with the K₁(nM) for the displacement of [³H] CP55940 from whole rat brain membranes. Efficacy at these as well as other G-protein-coupled receptors was measured as the capability of stimulating the binding of [³⁵S]GTPγS to rat hippocampal membranes. Potency (nM) and efficacy (maximal effect as percentage of the stimulation of the effect of 4 μm capsaicin) at human VR₁ receptors were measured as the capability of enhancing [Ca²⁺]ᵢ via HEK-hVR₁ cells. Data for VR₁ and CB₁ are means ±S.D. of n=3. For GTPγS binding, data are shown as means and range of n=3. The Eₘₐₓ and EC₅₀ of the potent CB₁ agonist WIN55,212-2 in the GTPγS assay were 51% and 82 nM, respectively. For the CB₁/VR₁ hybrid agonist O-1861, Eₘₐₓ and EC₅₀ were 32% and 70 nM, respectively. The capability of the compounds to interact with the AMT and FAAH was assessed by determining their inhibitory effect on [¹⁴C] AEA uptake by RBL-2H3 cells, where the AMT has been well characterized, and on [¹⁴C] AEA hydrolysis by rat brain membranes, where FAAH is quite abundant and uniquely responsible for anandamide degradation. The inhibitory effects are expressed as IC₅₀ and represent means ±S.D., n=3.

TABLE 3

| | | GTP_{γ}S | hVR₁ | AEA | |
|---|---|---|---|---|---|
| | CB₁ affinity | binding | potency | transport | FAAH |
| Comp. | Kᵢ | (Eₘₐₓ) | (EC₅₀) | (IC₅₀) | (IC₅₀) |
| 0-1988 | 2829 ± 175 | 0 | 25.0 ± 3.9 | 10.0 ± 2.1 | >50 |
| | | | (69.1 ± 5.2) | | |
| 0-1986 | 484 ± 17 | 0 | 63.0 ± 10.1 | 27.3 ± 3.5 | 18.2 ± 2.8 |
| | | | (68.0 ± 4.3) | | |
| 0-2094 | 274 ± 19 | 15 | 10 ± 1.4 | 19.0 ± 2.1 | 4.5 ± 0.7 |
| | | (8 of 20) | (63.5 ± 5.3) | | |
| 0-1987 | 1718 ± 200 | 0 | 0.7 ± 0.2 | 19.3 ± 3.3 | 2.0 ± 0.4 |
| | | | (80.6 ± 8.3) | | |
| 0-2095 | 8626 ± 130 | 0 | 0.4 ± 0.1 | 7.5 ± 1.8 | >50 |
| | | | (72.1 ± 4.8) | | |
| 0-2109 | 1801 ± 204 | 0 | 4.0 ± 1.1 | 3.8 ± 0.7 | >50 |
| 0-2142 | 483 ± 63 | 0 | 0.6 ± 0.2 | 30.0 ± 4.3 | 9.1 ± 1.7 |
| | | | (86.6 ± 8.2) | | |

Table 3 shows that all compounds were found to exhibit low CB₁ receptor affinity and little efficacy for stimulating G-protein coupling. The methylation of the amide in arvanil (O-1988) led to dramatic decreases in both the affinity and functional activity for CB₁ receptors. Deletion of the p-hydroxy-group on the aromatic moiety (O-1986) resulted in similar decreases, although some affinity for CB₁ receptors was retained. Substitution of an m-chloro for the m-methoxy in arvanil led to O-2094, a compound that had reasonable CB₁ receptor affinity and stimulated GTPγS binding [Eₘₐₓ=15% stimulation (8-20), EC₅₀=131 nM (5-1900), reversed by 2 nM SR141716A] slightly less potently than arvanil.

Conversion of the amide group in arvanil to a urea in O-1987 decreased both CB₁ affinity and GTPγS binding activity. When the urea was changed to a thiourea (O-2095) CB₁ receptor affinity was reduced further. The introduction of a chlorine atom in O-2095, which yielded O-2109, the thiourea analogue of O-2094, increased the activity of this compound in the CB₁ binding assay, but did not restore the activity in the GTPγS binding assay (Table 3). Finally, introduction of a 4′-morpholinobutyryl group on thep-hydroxy-benzyl group of arvanil, which yielded the water-soluble compound O-2142, again did not greatly influence the affinity for CB₁ receptors. This may be the result of the hydrolysis of the ester bond during the binding assay. However, it should be noted that: 1) O-2142 was inactive in the GTPγS binding assay, which is performed with rat brain membranes, and 2) a similar chemical modification in (R)-methanandamide decreased of about 20-fold its affinity for CB₁ receptors (from 20 nM to 426 nM, data not shown).

All the compounds tested except one were quite potent and efficacious in the functional assay of VR₁ activity performed in this study (Table 3), where the capability of increasing the [Ca²⁺]ᵢ was measured in HEK cells over-expressing the hVR₁ receptor. In agreement with previous structure/activity relationship studies carried out with capsaicin analogues and rat native vanilloid receptors (see, e.g., Walpole et al., J. Med. Chem. 36:2362-2372 (1993)), the substitution of the m-methoxy group with a chlorine atom (O-2094) and the methylation of the amide group (O-1988) in arvanil decreased its potency at hVR₁ by 25-50-fold. Introduction of a cc amide group (O-1987), and further substitution of the carbonyl for a C═S group (O-2095) did not alter arvanil efficacy/potency at VR₁. The importance of the p-hydroxy-benzyl group for the functional activation of these receptors is underlined by the observation that O-1986 was more than 100-fold less potent than arvanil, whereas the role of the m-methoxy group in the correct interaction with VR₁ was confirmed by the finding that O-2109 was 10-fold less potent than O-2095 (Table 3). Finally, and surprisingly, O-2142 was as potent as arvanil in inducing a VR₁-mediated increase of [Ca²⁺]ᵢ in HEK-hVR₁ cells. Since (i) this compound did not appear to be a good substrate for the AMT (see below), (ii) AMT-mediated facilitated transport into HEK-hVR₁ cells is important to observe high potency at hVR₁, and (iii) the p-hydroxy group is fundamental for interaction with vanilloid receptors, this finding suggests that O-2142 is hydrolysed by cells prior to its interaction with VR₁.

The overlap between the ligand recognition properties of the VR₁ receptor and the AMT, is supported by the observations with the current analogs (Table 3). Indeed, the order of potency of the compounds as AMT inhibitors (O-2109>O-2095>O-1988>O-2094=O-1987>O-1986>O-2142) was slightly different from the order of potency at hVR₁ (O-2095≧O-2142≧O-1987>O-2109>O-2094>O-1988>O-1986), although in most cases the differences between the activities of the compounds were not significant. However, if one excludes O-2142, whose activity at VR₁ might have been due to hydrolysis to arvanil, one of the compounds (O-1986) with the lowest activity on the AMT exhibited also low potency at VR₁, whereas O-2095 and O-2109 were quite potent as both VR₁ agonists and AMT inhibitors. In fact, the IC₅₀ of the widely used AMT inhibitor, AM404 (see, e.g., Khanolkar and Makriyannis, Life Sciences 65:607-616 (1999)) was 8.1±2.6 μM under the conditions set forth herein. This suggests that, if AMT inhibitors selective vs. the AMT are to be developed, they must contain either an o-methyl group on the vanillyl moiety, as in the case of VDM11, or hindering aromatic moieties, as in the case VDM13 and, possibly, other previously described arachidonate derivatives.

Unlike arvanil and its derivatives obtained through the modification of the aliphatic moiety, analogues obtained from the substitution of the m-methoxy group for a chlorine atom (O-2094), or from the introduction of an amide a to the carbonyl (O-1987), are potent FAAH inhibitors. Also elimination or derivatization of the p-hydroxy group, as in O-1986 and O-2142, respectively, slightly increases the affinity for FAAH. Given the esterase activity of FAAH, it is possible that the enzyme recognizes O-2142 as a better substrate due to the presence of the ester, rather than of the amide, bond. Conversely, modification of one of those chemical moieties that were previously shown to confer to AEA derivatives the capability of interacting with the enzyme, i.e. the carbonyl group, as in O-2095 vs. O-1987, abolished inhibitory activity (Table 3). The data also indicate that the carbonyl group is such a necessary requisite for interaction with FAAH that its elimination in O-1987 cannot be compensated for by the presence of the m-chlorine atom in the vanillyl moiety (O-2109). Another important requisite is the presence of a secondary or primary amide, and in fact O-1988 was even less potent inhibitors of FAAH activity than arvanil. Finally, the finding that O-1988, 0-2095 and O-2109 are all much more potent as AMT than as FAAH inhibitors, confirms that AEA transport into cells is not uniquely driven by FAAH activity as substances that inhibit this process without significantly affecting AEA hydrolysis can be found.

Table 4 shows the effect of arvanil derivatives in the mouse tetrad of behavioral tests. The ED₅₀ (milligrams per kilogram, i.v.) does for inhibition of sector crossings (spontaneous activity) in an open field, antinociception (delay in seconds in the tail-flick response), decrease of rectal temperature in degrees Celcius, and induction of time spent in immobility on a ring are shown. Data are means (and ranges) of N>six animals.

TABLE 4

| | Spontan. | | | | |
|---|---|---|---|---|---|
| Comp. | activity | Tail Flick | Rectal temp. | R.I. | Average |
| 0-1988 | 3.11 (1.54-6.29) | 7.98 (5.17-12.34) | 8.74 (5.61-13.60) | 4.08 (2.99-5.56) | 5.98 |
| 0-1986 | 4.74 (3.17-7.09) | 6.15 (4.63-8.16) | 6.15 (3.22-11.73) | 6.13 (4.79-7.83) | 5.80 |
| 0-2094 | 0.52 (0.38-0.71) | 0.49 (0.35-0.67) | 3.77 (1.21-11.76) | 1.93 (1.56-2.39) | 1.68 |
| 0-1987 | 0.06 (0.04-0.08) | 0.12 (0.08-0.17) | 0.08 (0.03-0.20) | 0.09 (0.06-0.12) | 0.09 |
| 0-2095 | 0.02 (0.02-0.03) | 0.08 (0.06-0.10) | 0.03 (0.02-0.07) | 0.08 (0.05-0.12) | 0.05 |
| 0-2109 | 0.02 (0.02-0.03) | 0.09 (0.06-0.12) | 0.33 (0.02-5.06) | 0.15 (0.10-0.23) | 0.15 |
| 0-2142 | 0.20 (0.16-0.24) | 0.19 (0.15-0.25) | 0.11 (0.07-0.17) | 0.17 (0.13-0.22) | 0.17 |

Of the eight compounds tested in this study, only those with a threshold potency at VR₁ receptors of 10 nM exhibited very strong activity (average ED₅₀ <1 mg/kg) in the mouse “tetrad” of tests (Table 4). Usually, a positive response (inhibition of locomotor activity, induction of immobility, antinociception and hypothermnia) in all four tests is considered highly indicative of cannabimimetic activity (Martin et al., Pharmacol. Biochem. Behavior, 40:471-478 (1991)). Yet, none of the arvanil analogues tested here bound with very high affinity to CB₁ receptors nor exhibited high efficacy in the GTPγS binding assay. Conversely, they displayed very high potency/efficacy at human VR₁ receptors, although their EC₅₀ values for VR₁ activation did not appear to correlate linearly with the ED₅₀ values observed in vivo. Finally, as best shown in Table 5 as well as in data not shown, the effect of O-2094 (either 1 or 3 mg/kg, i.v.) in the spontaneous activity, tail-flick and rectal temperature tests, and of O-1988 (either 3 or 10 mg/kg, i.v.) in the spontaneous activity and tail-flick tests were not affected by 10 min pre-treatment of mice with SR141716A (3 mg/kg, i.v.).

Table 5 shows the lack of effect by SR14176A (3 mg/kg administered i.v. ten minutes prior to compounds) on the hypocolomotor and antinociceptive effects of select arvanil derivatives in mice. No inhibitory effect by SR14176A was observed on the hypothermia induces by 0-2094 under similar conditions (not shown). Data are means ±S.E. of n≧six animals.

TABLE 5

| | | Spontaneous | | | |
|---|---|---|---|---|---|
| | | Activity | Tail-Flick Latency | | |
| | Dose | (Beam Interuptions) | (% MPE) | | |
| comp. | (mg/kg) | vehicle + compound | SR14176a + comp | vehicle + compound | SR14176a + comp |
| 0-1988 | 0 | 1224 ± 94 | 1188 ± 142 | 4 ± 2 | 21 ± 3 |
| | 3 | 766 ± 157 | 689 ± 68 | 54 ± 9 | 67 ± 15 |
| | 10 | 179 ± 34 | 117 ± 17 | 100 | 100 |
| 0-2094 | 0 | 1095 ± 93 | 2034 ± 182 | 9 ± 2 | 19 ± 5 |
| | 1 | 83 ± 17 | 116 ± 21 | 94 ± 5 | 100 |
| | 3 | 118 ± 16 | 52 ± 19 | 100 | 96 ± 4 |

None of the novel arvanil analogs described here bound to the CB₁ receptor with high affinity. However, some general conclusions emerge. Since O-2094 exhibited an affinity for CB₁ receptors similar to that previously observed for arvanil, it is possible to conclude that the presence of an m-methoxy group in the latter molecule is not crucial for the functional interaction with the central cannabinoid receptor. Conversely, the derivatization of the amide in arvanil, as in O-1988, and the lack of the para-hydroxy-group on the aromatic moiety, as in O-1986, led to dramatic changes in both the affinity for, and functional activity at, CB₁ receptors. In fact, previous studies showed that a secondary amide group in AEA derivatives is fundamental for interaction with cannabinoid receptors. These findings are also in agreement with previous data showing that the carbonyl function in AEA is important for the interaction with CB₁ binding site.

Although a certain overlap in the ligand recognition properties between CB₁ and VR₁ receptors can been observed, the chemical requisites for the optimal interaction of arvanil analogues with the binding sites within each receptor class are different. In particular, the p-hydroxy and m-methoxy groups on the vanillyl moiety are important for the interaction with VR₁ but not so much with CB₁ receptors; conversely, a carbonyl function on C-1 and a methylene group on C-2 in arvanil are important to achieve high affinity for CB₁ receptors but can be substituted for C═S and NH groups, respectively, without modifying the efficacy at VR₁. On the other hand, the presence of a 20 carbon atom polyunsaturated chain and of a secondary amide group are important for an optimal interaction with both receptor classes.

While the overlap between the AMT and VR1 ligand recognition properties is supported, to a great extent, by the present findings, rather surprising data emerged here on the capability of some of the novel analogs to inhibit FAAH. In general, it can be concluded that, despite the high potency of arvanil as an AMT inhibitor, the types of modifications of the amide and aromatic moieties made here on arvanil do not confer to this compound any further selectivity vs. VR1 or FAAH.

The observation that capsaicin exhibits a certain, albeit more limited, activity in some of the “tetrad” tests, that arvanil analogues are very potent in this mouse model, and an 18 carbon atom unsaturated capsaicin analog, livanil, inhibits locomotor activity in rats, might suggest that activation of VR₁ is also involved in inducing cannabimimetic responses in these assays. This suggestion is supported by the finding that the novel compounds with very high potency at hVR1 are also the most potent in the mouse “tetrad”. Since AEA also activates VR₁ receptors with a potency that may depend on several regulatory factors, it is possible that these sites also participate in AEA actions in the mouse “tetrad” tests, actions that cannot be reversed by a CB₁ receptor antagonist. Another possible explanation is that non-CB₁, non-VR₁ cannabinoid receptors are involved in the effects of arvanil-related compounds and, to some extent, of AEA in these four behavioural assays. In fact, several pharmacological actions of arvanil do not appear to be sensitive to effective doses of the CB₁ antagonist SR141716A or of the VR₁ antagonist capsazepine. In support to this, it has been found that the effects in some of the “tetrad” tests of three compounds with low and high potency, i.e. O-1988 and O-2094, respectively, were not antagonized by SR141716A.

### EXAMPLE 4

The arvanil analog O-2094 is characterized more fully herein. O-2094 had an affinity of 275±16 nM at the CB₁ receptor and was efficacious and potent in all four parameters of the mouse ‘tetrad’. O-2094 had ED₅₀ values ranging from 0.49-3.77 mg/kg. In order to determine whether this high potency was a result of an activity of the compound at VR₁ receptors, the ability of O-2094 to induce calcium influx in a human VR₁ transfected HEK cell line was tested. It was found that O-2094 was active in this assay (EC₅₀ =10 mM). Using the GTP_{γ}S binding assay (hippocampal membranes) to assess CB₁ activities of this compound, it was found that O-2094 acted as a low efficacy partial agonist, sensitive to antagonism by SR14176A (2 nM), and at a potency which correlated with CB₁ binding.

While the invention has been described in terms of its preferred embodiments, the invention can be practiced with modification and variation within the spirit and scope of the appended claims.

We claim:

1. A compound having the general structure: 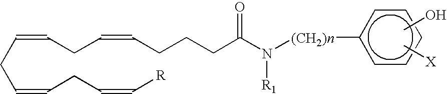
where n ranges from 0-5;
X represents a hydrogen, C₁₋₆ alkyl, halogen, hydroxy, or C₁₋₆ alkoxy;
R₁ represents hydrogen or C₁₋₆ alkyl; and
R is represented by the chemical structure 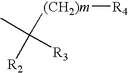
where m ranges from 1-7; R₂ and R₃ represent a hydrogen or C₁₋₆ alkyl group and may be the same or different from each other; and R₄ represents hydrogen, hydroxy, halogen, cyano (CN), C₁₋₆ alkyl (e.g., methyl (CH₃)), ONO, ONO₂, and NO₂.

2. The compound of claim 1 where the compound is selected from the group consisting of:

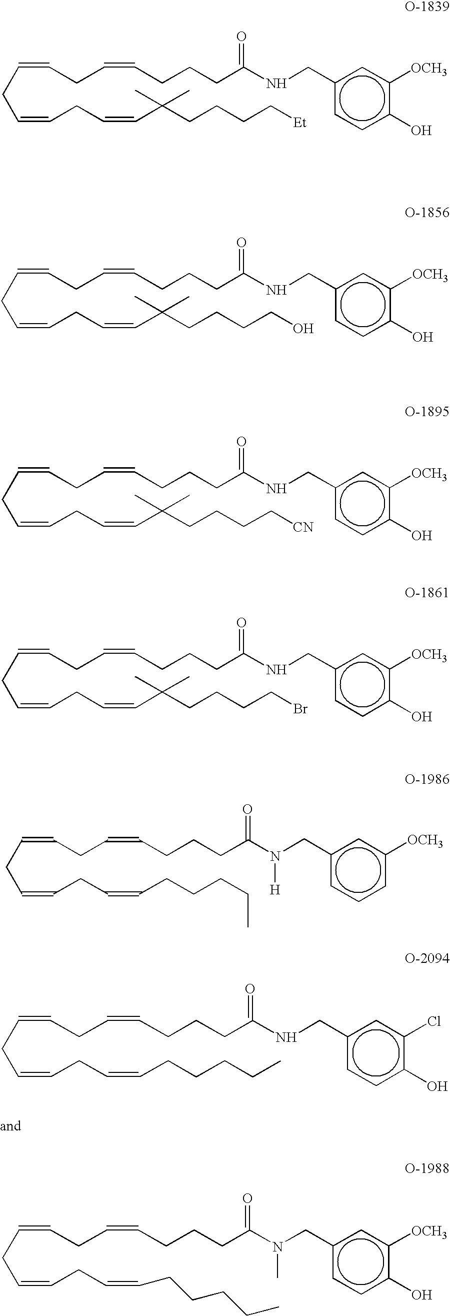

3. A compound having the following general structure: 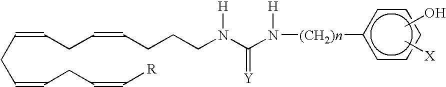
where n ranges from 0-3;
X represents a hydrogen, C₁₋₆ alkyl, halogen, hydroxy, and C₁₋₆ alkoxy;
Y represents S or O; and
R is represented by the chemical structure 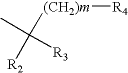
where m ranges from 1-7; R₂ and R₃ represent a hydrogen or C₁₋₆ alkyl group and may be the same or different from each other; and R₄ represents hydrogen, hydroxy, halogen, cyano (CN), C₁₋₆ alkyl (e.g., methyl (CH₃)), ONO, ONO₂, and NO₂.

4. The compound of claim 3 wherein in the compound is selected from the group consisting of:

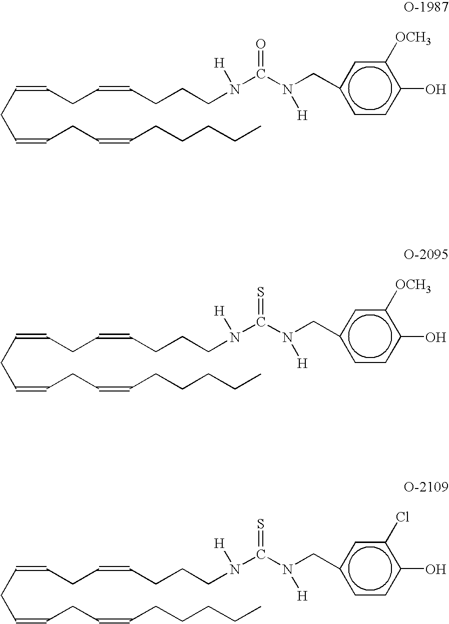

5. An analog of anandamide methylated at carbon 16 and having a chemical structure selected from the group consisting of:

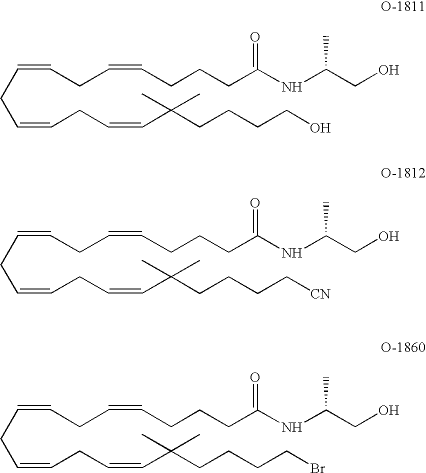

6. An analog of arvanil having a chemical structure selected from the group consisting of:

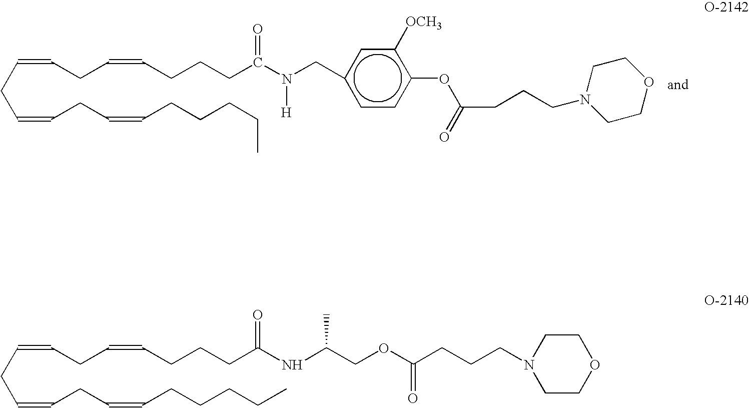

7. A method for selectively blocking CB₁ receptors in a cell or host without blocking CB₂ and VR₁ receptors, comprising the step of providing said cell or said host with a compound selected from the group consisting of

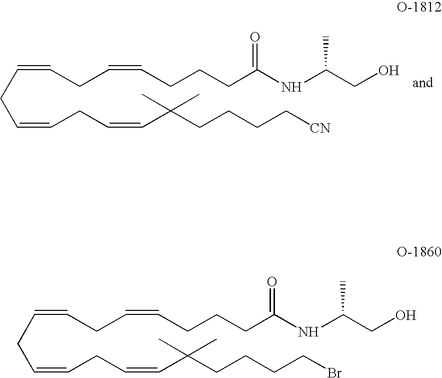

8. A method for increasing the potency of an anandamide or arvanil analog at CB₁ receptors, comprising the step of brominating or cyanating an anandamide or arvanil analog at a C-20 position of said anandamide or arvanil analog.

9. The method of claim 8, wherein said anandamid or arvanil analog is an arvanil analog and further comprising the step of methylating said arvanil analog at a C-16 position.

10. A method of managing pain in a patient in need thereof, comprising the step of
administering to said patient a compound as recited in any of claims 1, 3, 5 or 6, in a quantity sufficient to manage said pain.
